SEQUENCE LISTING (69 sequences)

SEQ ID NO: 1 - protein, 4 aa, synthetic construct
  REPEAT 1: note = Wherein Gly can be repeated between 1 to 4 times
  REGION 1..2: note = Linker
  REPEAT 1..2: note = Wherein the amino acids herein can be repeated 1 to 49 times.
  VARIANT 3..4: note = Wherein the amino acids can be present or not present
  VARIANT 2: note = Wherein Ser can be present or not present
  REPEAT 3: note = Wherein Gly can be repeated between 1 to 4 times
  VARIANT 4: note = Wherein Ser can be present or not present
GSGS

SEQ ID NO: 2 - RNA, 10 nt, synthetic construct
  misc_feature 1..10: note = Antisense Oligonucleotide
  source 1..10: mol_type = other RNA
ccgtgtatga

SEQ ID NO: 3 - RNA, 11 nt, synthetic construct
  misc_feature 1..11: note = Antisense Oligonucleotide
  source 1..11: mol_type = other RNA
gccgtgtatga

SEQ ID NO: 4 - RNA, 12 nt, synthetic construct
  misc_feature 1..12: note = Antisense Oligonucleotide
  source 1..12: mol_type = other RNA
agccgtgtatga

SEQ ID NO: 5 - RNA, 13 nt, synthetic construct
  misc_feature 1..13: note = Antisense Oligonucleotide
  source 1..13: mol_type = other RNA
gagccgtgtatga

SEQ ID NO: 6 - RNA, 14 nt, synthetic construct
  misc_feature 1..14: note = Antisense Oligonucleotide
  source 1..14: mol_type = other RNA
agagccgtgtatga

SEQ ID NO: 7 - RNA, 15 nt, synthetic construct
  misc_feature 1..15: note = Antisense Oligonucleotide
  source 1..15: mol_type = other RNA
gagagccgtgtatga

SEQ ID NO: 8 - RNA, 16 nt, synthetic construct
  misc_feature 1..16: note = Antisense Oligonucleotide
  source 1..16: mol_type = other RNA
ggagagccgtgtatga

SEQ ID NO: 9 - RNA, 17 nt, synthetic construct
  misc_feature 1..17: note = Antisense Oligonucleotide
  source 1..17: mol_type = other RNA
aggagagccgtgtatga

SEQ ID NO: 10 - RNA, 18 nt, synthetic construct
  misc_feature 1..18: note = Antisense Oligonucleotide
  source 1..18: mol_type = other RNA
gaggagagccgtgtatga

SEQ ID NO: 11 - RNA, 19 nt, synthetic construct
  misc_feature 1..19: note = Antisense Oligonucleotide
  source 1..19: mol_type = other RNA
agaggagagccgtgtatga

SEQ ID NO: 12 - RNA, 20 nt, synthetic construct
  misc_feature 1..20: note = Antisense Oligonucleotide
  source 1..20: mol_type = other RNA
gagaggagagccgtgtatga

SEQ ID NO: 13 - RNA, 10 nt, synthetic construct
  misc_feature 1..10: note = Antisense Oligonucleotide
  source 1..10: mol_type = other RNA
cgtgtatgac

SEQ ID NO: 14 - RNA, 11 nt, synthetic construct
  misc_feature 1..11: note = Antisense Oligonucleotide
  source 1..11: mol_type = other RNA
ccgtgtatgac

SEQ ID NO: 15 - RNA, 12 nt, synthetic construct
  misc_feature 1..12: note = Antisense Oligonucleotide
  source 1..12: mol_type = other RNA
gccgtgtatgac

SEQ ID NO: 16 - RNA, 13 nt, synthetic construct
  misc_feature 1..13: note = Antisense Oligonucleotide
  source 1..13: mol_type = other RNA
agccgtgtatgac

SEQ ID NO: 17 - RNA, 14 nt, synthetic construct
  misc_feature 1..14: note = Antisense Oligonucleotide
  source 1..14: mol_type = other RNA
gagccgtgtatgac

SEQ ID NO: 18 - RNA, 15 nt, synthetic construct
  misc_feature 1..15: note = Antisense Oligonucleotide
  source 1..15: mol_type = other RNA
agagccgtgtatgac

SEQ ID NO: 19 - RNA, 16 nt, synthetic construct
  misc_feature 1..16: note = Antisense Oligonucleotide
  source 1..16: mol_type = other RNA
gagagccgtgtatgac

SEQ ID NO: 20 - RNA, 17 nt, synthetic construct
  misc_feature 1..17: note = Antisense Oligonucleotide
  source 1..17: mol_type = other RNA
ggagagccgtgtatgac

SEQ ID NO: 21 - RNA, 18 nt, synthetic construct
  misc_feature 1..18: note = Antisense Oligonucleotide
  source 1..18: mol_type = other RNA
aggagagccgtgtatgac

SEQ ID NO: 22 - RNA, 19 nt, synthetic construct
  misc_feature 1..19: note = Antisense Oligonucleotide
  source 1..19: mol_type = other RNA
gaggagagccgtgtatgac

SEQ ID NO: 23 - RNA, 20 nt, synthetic construct
  misc_feature 1..20: note = Antisense Oligonucleotide
  source 1..20: mol_type = other RNA
agaggagagccgtgtatgac

SEQ ID NO: 24 - RNA, 21 nt, synthetic construct
  misc_feature 1..21: note = Antisense Oligonucleotide
  source 1..21: mol_type = other RNA
gagaggagagccgtgtatgac

SEQ ID NO: 25 - RNA, 22 nt, synthetic construct
  misc_feature 1..22: note = Antisense Oligonucleotide
  source 1..22: mol_type = other RNA
agagaggagagccgtgtatgac

SEQ ID NO: 26 - DNA, 10 bp, synthetic construct
  misc_feature 1..10: note = Antisense Oligonucleotide
  source 1..10: mol_type = other DNA
ccgtgtatga

SEQ ID NO: 27 - DNA, 11 bp, synthetic construct
  misc_feature 1..11: note = Antisense Oligonucleotide
  source 1..11: mol_type = other DNA
gccgtgtatga

SEQ ID NO: 28 - DNA, 12 bp, synthetic construct
  misc_feature 1..12: note = Antisense Oligonucleotide
  source 1..12: mol_type = other DNA
agccgtgtatga

SEQ ID NO: 29 - DNA, 13 bp, synthetic construct
  misc_feature 1..13: note = Antisense Oligonucleotide
  source 1..13: mol_type = other DNA
gagccgtgtatga

SEQ ID NO: 30 - DNA, 14 bp, synthetic construct
  misc_feature 1..14: note = Antisense Oligonucleotide
  source 1..14: mol_type = other DNA
agagccgtgtatga

SEQ ID NO: 31 - DNA, 15 bp, synthetic construct
  misc_feature 1..15: note = Antisense Oligonucleotide
  source 1..15: mol_type = other DNA
gagagccgtgtatga

SEQ ID NO: 32 - DNA, 16 bp, synthetic construct
  misc_feature 1..16: note = Antisense Oligonucleotide
  source 1..16: mol_type = other DNA
ggagagccgtgtatga

SEQ ID NO: 33 - DNA, 17 bp, synthetic construct
  misc_feature 1..17: note = Antisense Oligonucleotide
  source 1..17: mol_type = other DNA
aggagagccgtgtatga

SEQ ID NO: 34 - DNA, 18 bp, synthetic construct
  misc_feature 1..18: note = Antisense Oligonucleotide
  source 1..18: mol_type = other DNA
gaggagagccgtgtatga

SEQ ID NO: 35 - DNA, 19 bp, synthetic construct
  misc_feature 1..19: note = Antisense Oligonucleotide
  source 1..19: mol_type = other DNA
agaggagagccgtgtatga

SEQ ID NO: 36 - DNA, 20 bp, synthetic construct
  misc_feature 1..20: note = Antisense Oligonucleotide
  source 1..20: mol_type = other DNA
gagaggagagccgtgtatga

SEQ ID NO: 37 - DNA, 10 bp, synthetic construct
  misc_feature 1..10: note = Antisense Oligonucleotide
  source 1..10: mol_type = other DNA
cgtgtatgac

SEQ ID NO: 38 - DNA, 11 bp, synthetic construct
  misc_feature 1..11: note = Antisense Oligonucleotide
  source 1..11: mol_type = other DNA
ccgtgtatgac

SEQ ID NO: 39 - DNA, 12 bp, synthetic construct
  misc_feature 1..12: note = Antisense Oligonucleotide
  source 1..12: mol_type = other DNA
gccgtgtatgac

SEQ ID NO: 40 - DNA, 13 bp, synthetic construct
  misc_feature 1..13: note = Antisense Oligonucleotide
  source 1..13: mol_type = other DNA
agccgtgtatgac

SEQ ID NO: 41 - DNA, 14 bp, synthetic construct
  misc_feature 1..14: note = Antisense Oligonucleotide
  source 1..14: mol_type = other DNA
gagccgtgtatgac

SEQ ID NO: 42 - DNA, 15 bp, synthetic construct
  misc_feature 1..15: note = Antisense Oligonucleotide
  source 1..15: mol_type = other DNA
agagccgtgtatgac

SEQ ID NO: 43 - DNA, 16 bp, synthetic construct
  misc_feature 1..16: note = Antisense Oligonucleotide
  source 1..16: mol_type = other DNA
gagagccgtgtatgac

SEQ ID NO: 44 - DNA, 17 bp, synthetic construct
  misc_feature 1..17: note = Antisense Oligonucleotide
  source 1..17: mol_type = other DNA
ggagagccgtgtatgac

SEQ ID NO: 45 - DNA, 18 bp, synthetic construct
  misc_feature 1..18: note = Antisense Oligonucleotide
  source 1..18: mol_type = other DNA
aggagagccgtgtatgac

SEQ ID NO: 46 - DNA, 19 bp, synthetic construct
  misc_feature 1..19: note = Antisense Oligonucleotide
  source 1..19: mol_type = other DNA
gaggagagccgtgtatgac

SEQ ID NO: 47 - DNA, 20 bp, synthetic construct
  misc_feature 1..20: note = Antisense Oligonucleotide
  source 1..20: mol_type = other DNA
agaggagagccgtgtatgac

SEQ ID NO: 48 - DNA, 21 bp, synthetic construct
  misc_feature 1..21: note = Antisense Oligonucleotide
  source 1..21: mol_type = other DNA
gagaggagagccgtgtatgac

SEQ ID NO: 49 - DNA, 22 bp, synthetic construct
  misc_feature 1..22: note = Antisense Oligonucleotide
  source 1..22: mol_type = other DNA
agagaggagagccgtgtatgac

SEQ ID NO: 50 - RNA, 22 nt, synthetic construct
  misc_feature 1..22: note = human miR-204-5p
  source 1..22: mol_type = other RNA
ttccctttgtcatcctatgcct

SEQ ID NO: 51 - RNA, 22 nt, synthetic construct
  misc_feature 1..22: note = Antisense Oligonucleotide
  source 1..22: mol_type = other RNA
aggcataggatgacaaagggaa

SEQ ID NO: 52 - RNA, 22 nt, synthetic construct
  misc_feature 1..22: note = human miR-132-3p
  source 1..22: mol_type = other RNA
taacagtctacagccatggtcg

SEQ ID NO: 53 - RNA, 22 nt, synthetic construct
  misc_feature 1..22: note = Antisense Oligonucleotide
  source 1..22: mol_type = other RNA
cgaccatggctgtagactgtta

SEQ ID NO: 54 - RNA, 22 nt, synthetic construct
  misc_feature 1..22: note = human miR-485-3p
  source 1..22: mol_type = other RNA
gtcatacacggctctcctctct

SEQ ID NO: 55 - RNA, 22 nt, synthetic construct
  source 1..22: mol_type = other RNA
  misc_difference order(15,17,19): note = Thymine or Uracil
agagaggagagccgtgtatgac

SEQ ID NO: 56 - RNA, 22 nt, synthetic construct
  source 1..22: mol_type = other RNA
  misc_difference order(6,11): note = Thymine or Uracil
aggcataggatgacaaagggaa

SEQ ID NO: 57 - protein, 4 aa, synthetic construct
  REGION 1..2: note = Linker
  REPEAT 1: note = Wherein Gly can be repeated between 1 and 100 times
  REPEAT 1..2: note = Wherein the amino acids may be repeated between 1 to 99 times
  VARIANT 3..4: note = Wherein the amino acids can be present or not present
  REPEAT 3: note = wherein Gly can be repeated between 1 to 100 times
GSGS

SEQ ID NO: 58 - protein, 4 aa, synthetic construct
  REGION 1: note = Linker
  REPEAT 1: note = Wherein Gly may be repeated 1 to 97 times
  VARIANT 2: note = Wherein Gly can be present or not present
  VARIANT 3: note = Wherein Gly can be present or not present
  VARIANT 4: note = Wherein Gly can be present or not present
GGGG

SEQ ID NO: 59 - protein, 4 aa, synthetic construct
  REGION 1..4: note = Linker
GGGG

SEQ ID NO: 60 - protein, 4 aa, synthetic construct
  REGION 1..2: note = Linker
  REPEAT 1..2: note = Wherein the amino acids may be repeated between 1 and 99 times.
  VARIANT 3..4: note = Wherein the amino acids can be present or not present
GAGA

SEQ ID NO: 61 - protein, 6 aa, synthetic construct
  REGION 1..3: note = Linker
  REPEAT 1..3: note = Wherein the amino acids may be repeated between 1 and 99 times.
  VARIANT 4..6: note = Wherein the amino acids can be present or not present
GGSGGS

SEQ ID NO: 62 - protein, 4 aa, synthetic construct
  REGION 1..4: note = Linker
  REPEAT 1..4: note = Wherein the amino acids may be repeated between 1 to 100 times.
GGGS

SEQ ID NO: 63 - protein, 8 aa, synthetic construct
  REGION 1..8: note = linker
  REPEAT 1..3: note = Wherein the amino acids may be repeated between 1 to 100 times.
  REPEAT 4..8: note = Wherein the amino acids may be repeated between 1 to 100 times.
GGSGGGGS

SEQ ID NO: 64 - protein, 7 aa, synthetic construct
  REGION 1..7: note = Linker
SGGSGGS

SEQ ID NO: 65 - protein, 15 aa, synthetic construct
  REGION 1..15: note = Linker
GGSGGSGGSGGSGGG

SEQ ID NO: 66 - protein, 16 aa, synthetic construct
  REGION 1..16: note = Linker
GGSGGSGGGGSGGGGS

SEQ ID NO: 67 - protein, 18 aa, synthetic construct
  REGION 1..18: note = Linker
GGSGGSGGSGGSGGSGGS

SEQ ID NO: 68 - protein, 15 aa, synthetic construct
  REGION 1..15: note = Linker
GGGGSGGGGSGGGGS

SEQ ID NO: 69 - protein, 4 aa, synthetic construct
  REGION 1..4: note = Linker
  REPEAT 1..4: note = Wherein the amino acids may be repeated between 1 to 100 times.
GGGG

(19) United States
(12) Patent Application Publication
(10) Pub. No.: US 2024/0216529 A1
(43) Pub. Date: Jul. 4, 2024

(54) EXTRACELLULAR VESICLES WITH TARGETING MOIETY

(71) Applicant: BIORCHESTRA CO., LTD., Daejeon (KR)

(72) Inventor: Jin-Hyeob RYU, Daejeon (KR)

(73) Assignee: BIORCHESTRA CO., LTD., Daejeon (KR)

(21) Appl. No.: 18/145,352

(22) Filed: Dec. 22, 2022

(51) Int. Cl. A61K 47/69; C12N 15/113

(52) U.S. Cl. CPC A61K 47/6913; C12N 15/113; C12N 2310/141

(57) ABSTRACT

The present disclosure relates to extracellular vesicles (e.g., exosomes) comprising a targeting moiety and a biologically active molecule via an optional linker, which may be useful as an agent for the prophylaxis or treatment of diseases. Also provided herein are methods for producing the extracellular vesicles and methods for using the extracellular vesicles to treat diseases or disorders.

The content of the electronically submitted sequence listing (Name: 4366_0720000_Seqlisting_ST26.xml, Size 96,734 bytes; and Date of Creation: Oct. 17, 2023) submitted in this application is incorporated herein by reference in its entirety.

### TECHNICAL FIELD

The present disclosure provides extracellular vesicles (EVs), e.g., exosomes, which can be useful as an agent for the prophylaxis or treatment of cancer and other diseases comprising at least one targeting moiety.

### BACKGROUND

The blood-brain barrier restricts the passage of pathogens, the diffusion of solutes in the blood, and large or hydrophilic molecules into the cerebrospinal fluid (CSF), while allowing the diffusion of O₂, CO₂, hydrophobic molecules (e.g., hormones), and small polar molecules (Johansen et al., (2017) Journal of Cerebral Blood Flow and Metabolism. Epub (4): 659-668). The BBB excludes from the brain almost 100% of large-molecule neurotherapeutics and more than 98% of all molecule drugs. Daneman & Prat (2015) “The Blood Brain Barrier” Cold Spring Harbor Perspectives in Biology 7(1): a020412. Overcoming the difficulty of delivering therapeutic agents to specific regions of the brain represents a major challenge to treatment of most brain disorders. Thus, therapeutic molecules that might otherwise be effective in diagnosis and therapy do not cross the BBB in adequate amounts.

Intracellular targeting is also often challenging, because to reach the cytosol, exogenous molecules must first traverse the cell membrane. The cell membrane is selectively permeable to non-polar therapeutic agents, which are lipid soluble and can pass through the cell membrane. On the other hand, highly charged therapeutic agents such as oligonucleotides are effectively excluded by the cell membrane.

Thus, the problems facing the delivery of a payload, e.g., antisense oligonucleotide, can roughly be divided into two parts. First, the therapeutic payload must be formulated in such a way that it can be delivered to the cytoplasm and second, the payload must reach the cell nucleus intact and fully functional. Despite the advances in application of proteins, small molecules, and nucleotides as therapeutics, the need exists for delivery systems providing improved pharmacological properties, e.g., serum stability, delivery to the right organ, tissue, or cell, and transmembrane delivery.

### BRIEF SUMMARY

The present disclosure provides an extracellular vesicle (EV) comprising a targeting moiety via an optional linker, wherein the targeting moiety is capable of being transported by large neutral amino acid transporter 1 (LAT1). In some aspects, the extracellular vesicle further comprises a biologically active molecule. In some aspects, the biologically active molecule delivered by the EV is linked to the exterior surface of the EV. In some aspects, the biologically active molecule is inside of the EV. In some aspects, the biologically active molecule is linked to the interior surface of the EV.

In some aspects, the biologically active molecule delivered by the EV comprises a polypeptide, a peptide, a polynucleotide (DNA and/or RNA), a chemical compound, or any combination thereof. In some aspects, the biologically active molecule is a chemical compound. In some aspects, the chemical compound is a small molecule.

In some aspects, the biologically active molecule comprises a nucleic acid. In some aspects, the nucleic acid comprises a mRNA, a miRNA, a miRNA sponge, a tough decoy miRNA (TD), an antimir (antagomir), a small RNA, a rRNA, a siRNA, a shRNA, a gDNA, a cDNA, a pDNA, a PNA, a BNA, an antisense oligonucleotide (ASO), an aptamer, a cyclic dinucleotide, a phosphorodiamidate morpholino oligonucleotide (PMO), or any combination thereof. In some aspects, the nucleic acid comprises an ASO. In some aspects, the nucleic acid comprises an siRNA.

In some aspects, the ASO targets an miRNA. In some aspects, the miRNA is miR-485-3p, miR-204-5p, or miR-132-3p. In some aspects, the nucleic acid has at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In some aspects, the nucleic acid targets miR-485-3p and has a sequence selected from the group consisting of: 5′-UGUAUGA-3′, 5′-GUGUAUGA-3′, 5′-CGUGUAUGA-3′, 5′-CCGUGUAUGA-3′ (SEQ ID NO: 2), 5′-GCCGUGUAUGA-3′ (SEQ ID NO: 3), 5′-AGCCGUGUAUGA-3′ (SEQ ID NO: 4), 5′-GAGCCGUGUAUGA-3′ (SEQ ID NO: 5), 5′-AGAGCCGUGUAUGA-3′ (SEQ ID NO: 6), 5′-GAGAGCCGUGUAUGA-3′ (SEQ ID NO: 7), 5′-GGAGAGCCGUGUAUGA-3′ (SEQ ID NO: 8), 5′-AGGAGAGCCGUGUAUGA-3′ (SEQ ID NO: 9), 5′-GAGGAGAGCCGUGUAUGA-3′ (SEQ ID NO: 10), 5′-AGAGGAGAGCCGUGUAUGA-3′ (SEQ ID NO: 11), 5′-GAGAGGAGAGCCGUGUAUGA-3′ (SEQ ID NO: 12); 5′-UGUAUGAC-3′, 5′-GUGUAUGAC-3′, 5′-CGUGUAUGAC-3′ (SEQ ID NO: 13), 5′-CCGUGUAUGAC-3′ (SEQ ID NO: 14), 5′-GCCGUGUAUGAC-3′ (SEQ ID NO: 15), 5′-AGCCGUGUAUGAC-3′ (SEQ ID NO: 16), 5′-GAGCCGUGUAUGAC-3′ (SEQ ID NO: 17), 5′-AGAGCCGUGUAUGAC-3′ (SEQ ID NO: 18), 5′-GAGAGCCGUGUAUGAC-3′ (SEQ ID NO: 19), 5′-GGAGAGCCGUGUAUGAC-3′ (SEQ ID NO: 20), 5′-AGGAGAGCCGUGUAUGAC-3′ (SEQ ID NO: 21), 5′-GAGGAGAGCCGUGUAUGAC-3′ (SEQ ID NO: 22), 5′-AGAGGAGAGCCGUGUAUGAC-3′ (SEQ ID NO: 23), 5′-GAGAGGAGAGCCGUGUAUGAC-3′ (SEQ ID NO: 24), or 5′-AGAGAGGAGAGCCGUGUAUGAC-3′ (SEQ ID NO: 25).

In some aspects, the nucleic acid has a sequence selected from the group consisting of: 5′-TGTATGA-3′, 5′-GTGTATGA-3′, 5′-CGTGTATGA-3′, 5′-CCGTGTATGA-3′ (SEQ ID NO: 26), 5′-GCCGTGTATGA-3′ (SEQ ID NO: 27), 5′-AGCCGTGTATGA-3′ (SEQ ID NO: 28), 5′-GAGCCGTGTATGA-3′ (SEQ ID NO: 29), 5′-AGAGCCGTGTATGA-3′ (SEQ ID NO: 30), 5′-GAGAGCCGTGTATGA-3′ (SEQ ID NO: 31), 5′-GGAGAGCCGTGTATGA-3′ (SEQ ID NO: 32), 5′-AGGAGAGCCGTGTATGA-3′ (SEQ ID NO: 33), 5′-GAGGAGAGCCGTGTATGA-3′ (SEQ ID NO: 34), 5′-AGAGGAGAGCCGTGTATGA-3′ (SEQ ID NO: 35), 5′-GAGAGGAGAGCCGTGTATGA-3′ (SEQ ID NO: 36); 5′-TGTATGAC-3′, 5′-GTGTATGAC-3′, 5′-CGTGTATGAC-3′ (SEQ ID NO: 37), 5′-CCGTGTATGAC-3′ (SEQ ID NO: 38), 5′-GCCGTGTATGAC-3′ (SEQ ID NO: 39), 5′-AGCCGTGTATGAC-3′ (SEQ ID NO: 40), 5′-GAGCCGTGTATGAC-3′ (SEQ ID NO: 41), 5′-AGAGCCGTGTATGAC-3′ (SEQ ID NO: 42), 5′-GAGAGCCGTGTATGAC-3′ (SEQ ID NO: 43), 5′-GGAGAGCCGTGTATGAC-3′ (SEQ ID NO: 44), 5′-AGGAGAGCCGTGTATGAC-3′ (SEQ ID NO: 45), 5′-GAGGAGAGCCGTGTATGAC-3′ (SEQ ID NO: 46), 5′-AGAGGAGAGCCGTGTATGAC-3′ (SEQ ID NO: 47), 5′-GAGAGGAGAGCCGTGTATGAC-3′ (SEQ ID NO: 48), and 5′-AGAGAGGAGAGCCGTGTATGAC-3′ (SEQ ID NO: 49).

In some aspects, the nucleic acid has a sequence at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 25) or 5′-AGAGAGGAGAGCCGTGTATGAC-3′ (SEQ ID NO: 49). In some aspects, the nucleic acid has a sequence that has at least 90% similarity to 5′-AGAGAGGAGAGCCGUGUAUGAC-3′ (SEQ ID NO: 25) or 5′-AGAGAGGAGAGCCGTGTATGAC-3′ (SEQ ID NO: 49). In some aspects, the nucleic acid comprises the nucleotide sequence 5′-AGAGAGGAGAGCCGUGUAUGAC-3′ (SEQ ID NO: 25) or 5′-AGAGAGGAGAGCCGTGTATGAC-3′ (SEQ ID NO: 49) with one substitution or two substitutions. In some aspects, the nucleic acid comprises the nucleotide sequence 5′-AGAGAGGAGAGCCGUGUAUGAC-3′ (SEQ ID NO: 25) or 5′-AGAGAGGAGAGCCGTGTATGAC-3′ (SEQ ID NO: 49). In some aspects, the nucleic acid comprises the nucleotide sequence 5′-AGAGAGGAGAGCCGUGUAUGAC-3′ (SEQ ID NO: 25).

In some aspects, the nucleic acid comprises at least one modified nucleotide. In some aspects, the at least one modified nucleotide is a locked nucleic acid (LNA), an unlocked nucleic acid (UNA), an arabino nucleic acid (ABA), a bridged nucleic acid (BNA), and/or a peptide nucleic acid (PNA). In some aspects, the nucleic acid comprises a backbone modification. In some aspects, the backbone modification is a phosphorodiamidate morpholino oligomer (PMO) and/or phosphorothioate (PS) modification.

In some aspects, the nucleic acid targets a transcript. In some aspects, the nucleic acid is linked to the EV via a linker.

In some aspects, the biologically active molecule comprises a peptide, a protein, an antibody or an antigen binding fragment thereof, a fusion protein, or any combination thereof. In some aspects, the antigen binding fragment thereof comprises scFv, (scFv)2, Fab, Fab′, F(ab′)2, F(ab1)2, Fv, dAb, and Fd fragment, diabodys, antibody-related polypeptide, or any fragment thereof.

In some aspects, the targeting moiety is an amino acid. In some aspects, the targeting moiety comprises a branched-chain or aromatic amino acid. In some aspects, the amino acid is valine, leucine, and/or isoleucine. In some aspects, the amino acid is tryptophan and/or tyrosine. In some aspects, the amino acid is phenylalanine. In some aspects, the targeting moiety is linked to the EV via a linker.

In some aspects, the EV further comprises an exosomal protein. In some aspects, the targeting moiety is linked to the exosomal protein. In some aspects, the EV is an exosome. In some aspects, the exosome is a native exosome.

The present disclosure provides a pharmaceutical composition comprising the extracellular vesicle and a pharmaceutically acceptable carrier.

The present disclosure provides a kit comprising the EV disclosed herein or the pharmaceutical composition disclosed herein and instructions for use.

The present disclosure also provides a cell that produces the extracellular vesicle disclosed herein.

The present disclosure provides a method of producing the extracellular disclosed herein comprising culturing the cell in suitable condition.

The present disclosure provides a method of treating or preventing a disease or disorder in a subject in need thereof comprising administering the EV disclosed herein or the pharmaceutical composition disclosed herein to the subject. In some aspects, the disease or disorder is a cancer, an inflammatory disorder, a neurodegenerative disorder, a central nervous disease, or a metabolic disease. In some aspects, the EV is administered intravenously, intraperitoneally, nasally, orally, intramuscularly, subcutaneously, parenterally, or intratumorally.

### DETAILED DESCRIPTION

The present disclosure is directed to extracellular vesicles (EVs), e.g., exosomes, comprising at least one targeting moiety that is capable of being transported by large neutral amino acid transporter 1 (LAT1). The disclosure also provides extracellular vesicles (EVs), e.g., exosomes, comprising at least one targeting moiety and a biologically active molecule. Non-limiting examples of the various aspects are shown in the present disclosure.

Before the present disclosure is described in greater detail, it is to be understood that this invention is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)2, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “biologically active molecule” as use herein refers to any molecule that can be attached to an EV, e.g., exosome, via an anchoring moiety, wherein the molecule can have a therapeutic or prophylactic effect in a subject in need thereof, or be used for diagnostic purposes. Accordingly, by way of example, the term biologically active molecule includes proteins (e.g., antibodies, proteins, polypeptides, and derivatives, fragments, and variants thereof), lipids and derivatives thereof, carbohydrates (e.g., glycan portions in glycoproteins), or small molecules. In some aspects, the biologically active molecule is a radioisotope. In some aspects, the biologically active molecule is a detectable moiety, e.g., a radionuclide, a fluorescent molecule, or a contrast agent. In some aspects, the biologically active molecule can be or can comprise a targeting moiety or a tropism moiety. In some aspects, the biologically active molecule can be or can comprise, for example, an affinity ligand such as biotin. In some aspects, the biologically active molecule can be or can comprise a moiety capable to improve a pharmacokinetic or pharmacodynamic property, for example, a moiety capable in increase plasma half-life, e.g., a PEG moiety.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

As used herein, the term “exosomal protein” means a protein previously known to be enriched in EVs. In some aspects, “exosomal protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

The terms “excipient” and “carrier” are used interchangeably and refer to an inert substance added to a pharmaceutical composition to further facilitate administration of a compound.

As used herein, the terms “extracellular vesicle,” “EV,” and grammatical variants thereof, are used interchangeably and refer to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In some aspects, an exosome comprises a targeting moiety. As described infra, exosome can be derived from a producer cell, and isolated from the producer cell based on its size, density, biochemical parameters, or a combination thereof. In some aspects, the exosomes of the present disclosure are produced by cells that express one or more transgene products.

In some aspects, EVs, e.g., exosomes, e.g., nanovesicles, of the present disclosure are engineered to express a targeting moiety such that at least one biologically active molecule (e.g., a protein such as an antibody or ADC, a RNA or DNA such as an antisense oligonucleotide, a small molecule drug, a toxin) can be delivered by the EV, e.g., exosome, e.g., nanovesicle, to target a LAT-1.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired EVs (e.g., a plurality of EVs of known or unknown amount and/or concentration), that has undergone one or more processes of purification, e.g., a selection or an enrichment of the desired EV, e.g., exosome, preparation. In some aspects, isolating or purifying as used herein is the process of removing, partially removing (e.g., a fraction) of the EVs, e.g., exosomes, from a sample containing producer cells. In some aspects, an isolated EV, e.g., exosome, composition has no detectable undesired activity or, alternatively, the level or amount of the undesired activity is at or below an acceptable level or amount. In other aspects, an isolated EV, e.g., exosome, composition has an amount and/or concentration of desired EVs, e.g., exosomes, at or above an acceptable amount and/or concentration. In other aspects, the isolated EVs, e.g., exosome, composition is enriched as compared to the starting material (e.g., producer cell preparations) from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, isolated EV, e.g. exosome, preparations are substantially free of residual biological products. In some aspects, the isolated EV, e.g., exosome, preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites. Substantially free of residual biological products can also mean that the EV, e.g., exosome, composition contains no detectable producer cells and that only EVs, e.g., exosomes, are detectable.

The terms “linked,” “fused,” and grammatical variants thereof are used interchangeably and refer to a first moiety, e.g., a first amino acid sequence or nucleotide sequence, covalently or non-covalently joined to a second moiety, e.g., a second amino acid sequence, nucleotide sequence, and/or a lipid (e.g., cholesterol), respectively. The first moiety can be directly joined or juxtaposed to the second moiety or alternatively an intervening moiety can covalently join the first moiety to the second moiety. The term “linked” means not only a fusion of a first moiety to a second moiety at the C-terminus or the N-terminus, but also includes insertion of the whole first moiety (or the second moiety) into any two points, e.g., amino acids, in the second moiety (or the first moiety, respectively). In one aspect, the first moiety is linked to a second moiety by a peptide bond or a linker. The first moiety can be linked to a second moiety by a phosphodiester bond or a linker. The linker can be a peptide or a polypeptide (for polypeptide chains) or a nucleotide or a nucleotide chain (for nucleotide chains) or any chemical moiety (for polypeptide or polynucleotide chains or any chemical molecules). The term “linked” is also indicated by a hyphen (-).

The term “modified,” when used in the context of EVs, e.g., exosomes, described herein, refers to an alteration or engineering of an EV, e.g., exosome and/or its producer cell, such that the modified EV, e.g., exosome, is different from a naturally-occurring EV, e.g., exosome. In some aspects, a modified EV, e.g., exosome, described herein comprises a membrane that differs in composition of a protein, a lipid, a small molecular, a carbohydrate, etc. compared to the membrane of a naturally-occurring EV, e.g., exosome. E.g., the membrane comprises higher density or number of natural EV, e.g., exosome, proteins and/or membrane comprises proteins that are not naturally found in EV, e.g., exosomes. In certain aspects, such modifications to the membrane change the exterior surface of the EV, e.g., exosome.

As used herein the terms “modified protein” or “protein modification” refers to a protein having at least 15% identity to the non-mutant amino acid sequence of the protein. A modification of a protein includes a fragment or a variant of the protein. A modification of a protein can further include chemical, or physical modification to a fragment or a variant of the protein.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., an EV, such as exosome of the present disclosure, mixed or intermingled with, or suspended in one or more other chemical components, such as pharmaceutically-acceptable carriers and excipients. One purpose of a pharmaceutical composition is to facilitate administration of preparations of EVs, e.g., exosomes, to a subject.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV, e.g., exosome, via a linker, spacer, or combination thereof disclosed herein is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide (e.g., an antisense oligonucleotide, ASO).

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV, e.g., exosome, via a linker, spacer, or combination thereof disclosed herein is a polypeptide, e.g., an antibody or a derivative thereof such as an ADC, a PROTAC, a toxin, a fusion protein, or an enzyme.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

Unless otherwise indicated, reference to a compound that has one or more stereocenters intends each stereoisomer, and all combinations of stereoisomers, thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the term “substantially free” means that the sample comprising EVs, e.g., exosomes, comprises less than 10% of macromolecules, e.g., contaminants, by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein the term “therapeutically effective amount” is the amount of reagent or pharmaceutical compound comprising an EV or exosome of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof. In one aspect, the term “treating” or “treatment” means inducing an immune response in a subject against an antigen.

As used herein, the term “variant” of a molecule (e.g., functional molecule, antigen, or targeting moiety) refers to a molecule that shares certain structural and functional identities with another molecule upon comparison by a method known in the art. For example, a variant of a protein can include a substitution, insertion, deletion, frame shift or rearrangement in another protein.

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, variants or derivatives include, e.g., modified polypeptides. In some aspects, variants or derivatives of, e.g., polypeptides, polynucleotides, lipids, glycoproteins, are the result of chemical modification and/or endogenous modification. In some aspects, variants or derivatives are the result of in vivo modification. In some aspects, variants or derivatives are the result of in vitro modification. In yet other aspects, variant or derivatives are the result of intracellular modification in producer cells.

Modifications present in variants and derivatives include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination.

### II. EVs (e.g., Exosomes) Comprising Targeting Moiety

Extracellular vesicles (EVs) typically have 20 nm to 1000 nm in diameter; e.g., exosomes, which are small extracellular vesicles, have typically 100-200 nm in diameter. EVs, e.g., exosomes, are composed of a limiting lipid bilayer and a diverse set of proteins and nucleic acids (Maas, S. L. N., et al., Trends. Cell Biol. 27(3):172-188 (2017)). EVs, e.g., exosomes, exhibit preferential uptake in discrete cell types and tissues, and their tropism can be directed by adding proteins to their surface that interact with receptors on the surface of target cells (Alvarez-Erviti, L., et al., Nat. Biotechnol. 29(4):341-345 (2011)).

Unlike antibodies, EVs (e.g., exosomes) can accommodate large numbers of molecules attached to their surface, on the order of thousands to tens of thousands of molecules per EV (e.g., exosome). EV (e.g., exosome)-targeting moiety thus represent a platform to deliver therapeutic compounds to specific cell types, tissues, or organs, so that overall systemic exposure to the compound is limited, in turn reducing off-target toxicity. In this respect, the present disclosure provides specific targeting moieties that can deliver a biologically active molecule (e.g., an ASO) to its target.

In some aspects, the targeting moiety and/or the biologically active molecule can be attached to the extracellular vesicle via a linker. In some aspects, the linker comprise a spacer. The term “spacer” as used herein refers to a chemical and/or peptidic moiety which is capable of covalently linking together two spaced moieties (e.g., a biologically active molecule or LAT1 targeting moiety) into a normally stable dipartate molecule. Generally, spacers as not cleavable. For example, a spacer can be an alkyl chain, or a polymeric chain formed by example by glycol or glycerol units.

In some aspects, the “linkers” of the present disclosure prevent the aggregation of biological moieties on the surface of the EV (e.g., exosome). In some aspects, the length of the optimized linker connecting the extracellular vesicles and a biologically active molecule is between about 2 nm and about 30 nm.

In some aspects, the length of the linker is about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, or about 30 nm. In some aspects, the length of the linker is at least about 2 nm, at least about 3 nm, at least about 4 nm, at least about 5 nm, at least about 6 nm, at least about 7 nm, at least about 8 nm, at least about 9 nm, at least about 10 nm, at least about 11 nm, at least about 12 nm, at least about 13 nm, at least about 14 nm, at least about 15 nm, at least about 16 nm, at least about 17 nm, at least about 18 nm, at least about 19 nm, at least about 20 nm, at least about 21 nm, at least about 22 nm, at least about 23 nm, at least about 24 nm, at least about 25 nm, at least about 26 nm, at least about 27 nm, at least about 28 nm, at least about 29 nm, or at least about 30 nm. In some aspects, the length of the linker is less than about 2 nm, less than about 3 nm, less than about 4 nm, less than about 5 nm, less than about 6 nm, less than about 7 nm, less than about 8 nm, less than about 9 nm, less than about 10 nm, less than about 11 nm, less than about 12 nm, less than about 13 nm, less than about 14 nm, less than about 15 nm, less than about 16 nm, less than about 17 nm, less than about 18 nm, less than about 19 nm, less than about 20 nm, less than about 21 nm, less than about 22 nm, less than about 23 nm, less than about 24 nm, less than about 25 nm, less than about 26 nm, less than about 27 nm, less than about 28 nm, less than about 29 nm, or less than about 30 nm.

In some aspects, the length of the linker is about 2 nm to about 4 nm, about 3 nm to about 5 nm, about 4 nm to about 6 nm, about 5 nm to about 7 nm, about 6 nm to about 8 nm, about 7 nm to about 9 nm, about 8 nm to about 10 nm, about 9 nm to about 11 nm, about 10 nm to about 12 nm, about 11 nm to about 13 nm, about 12 nm to about 14 nm, about 13 nm to about 15 nm, about 14 nm to about 16 nm, about 15 nm to about 17 nm, about 16 nm to about 18 nm, about 17 nm to about 19 nm, about 18 nm to about 20 nm, about 19 nm to about 21 nm, about 20 nm to about 22 nm, about 21 nm to about 23 nm, about 22 nm to about 24 nm, about 23 nm to about 25 nm, about 24 nm to about 26 nm, about 25 nm to about 27 nm, about 26 nm to about 28 nm, about 27 nm to about 29 nm, about 28 nm to about 30 nm, about 2 nm to about 6 nm, about 4 nm to about 8 nm, about 6 nm to about 10 nm, about 8 nm to about 12 nm, about 10 nm to about 14 nm, about 12 nm to about 16 nm, about 14 nm to about 18 nm, about 16 nm to about 20 nm to about, about 18 nm to about 22 nm, about 20 nm to about 24 nm, about 22 nm to about 26 nm, about 24 nm to about 28 nm, about 26 nm to about 30 nm, about 2 nm to about 10 nm, about 4 nm to about 12 nm, about 6 nm to about 14 nm, about 8 nm to about 16 nm, about 10 nm to about 18 nm, about 12 nm to about 20 nm, about 14 nm to about 22 nm, about 16 nm to about 24 nm, about 18 nm to about 26 nm, about 20 nm to about 28 nm, about 22 nm to about 30 nm, about 2 nm to about 12 nm, about 4 nm to about 14 nm, about 6 nm to about 16 nm, about 8 nm to about 18 nm, about 10 nm to about 20 nm, about 12 nm to about 22 nm, about 14 nm to about 24 nm, about 16 nm to about 26 nm, about 18 nm to about 28 nm, about 20 nm to about 30 nm, about 2 nm to about 5 nm, about 5 nm to about 10 nm, about 10 nm to about 15 nm, about 15 nm to about 20 nm, about 20 nm to about 25 nm, or about 25 nm to about 30 nm.

In some aspects, the biologically active molecule comprises or consists of a polypeptide, a peptide, a polynucleotide (DNA and/or RNA), a chemical compound, or any combination thereof.

In some aspects, the biologically active molecule comprises a chemical compound, e.g., a small molecule drug. In some aspects, the biologically active molecule comprises an antisense oligonucleotide (ASO), phosphorodiamidate morpholino oligonucleotide (PMO), a siRNA, a miRNA, a shRNA, a nucleic acid, an antimirs, an RNA decoy, or any combination thereof. In some aspects, the ASO is a gapmer. In some aspects, the ASO targets miRNA. Specific nucleic acid biologically active molecules, e.g., ASOs against specific targets are disclosed in detail below.

In some aspects, the biologically active molecule comprises or consists of a peptide, a protein, an antibody or an antigen binding portion thereof, or any combination thereof. In some aspects, the antigen binding portion thereof comprises scFv, (scFv)2, Fab, Fab′, F(ab′)2, F(ab1)2, Fv, dAb, and Fd fragment, diabodys, antibody-related polypeptide, or any fragment thereof.

In some aspects, biologically active molecule comprises or consist of an ASO. In some aspects, the ASO can target miRNA, pre-mRNA or a mature mRNA, including protein coding regions (exons), non coding regions (e.g., 5′ or 3′ untranslated regions, or introns), intron-exon junctions, or regulatory regions (e.g., promoters). In some aspects, the ASO targets a protein transcript. Detailed descriptions of ASOs directed to specific targets are provided below.

The present disclose provides a method of treating or preventing a disease or disorder in a subject in need thereof comprising administering an EV (e.g., exosome) of the present disclosure or pharmaceutical composition comprising such EV (e.g., exosome) to the subject. In some aspects, the disease or disorder is a cancer, an inflammatory disorder, a neurodegenerative disorder, a central nervous disease, or a metabolic disease. In some aspects, an EV (e.g., exosome) disclosed herein can be administered intravenously, intraperitoneally, nasally, orally, intramuscularly, subcutaneously, parenterally, intrathecally, peritumorally, intraocularly, or intratumorally.

### IIA. Targeting Moiety

The extracellular vesicle of the present disclosure comprises a targeting moiety, which can be linked to the EV optionally via a linker. As used herein, the term “targeting moiety” refers to a biorecognition molecule that binds to a specific biological substance or site. In some aspects, the targeting moiety is specific for LAT-1.

For targeting a payload (e.g., a nucleotide molecule, e.g., an ASO) according to the present disclosure, a targeting moiety can be coupled to the external surface of an EV, whereas the EV has the payload entrapped within its core.

In some aspects, the targeting moiety is a targeting moiety capable of targeting the EV of the present disclosure to a tissue that expresses LAT1. In some aspects, the tissue is liver, brain, kidney, lung, ovary, pancreas, thyroid, breast, stomach, or any combination thereof.

In some aspects, the tissue is a tissue in the central nervous system, e.g., neural tissue. In some aspects, the targeting moiety targeting the central nervous system is capable being transported by Large-neutral Amino Acid Transporter 1 (LAT1). LAT1 (SLC7A5) is a transporter for both the uptake of large neutral amino acids and a number of pharmaceutical drugs. LAT1 can transport drugs such as L-dopa or gabapentin.

In some aspects, a targeting moiety comprises glucose, e.g., D-glucose, which can bind to Glucose transporter 1 (or GLUT1) and cross BBB. GLUT1, also known as solute carrier family 2, facilitated glucose transporter member 1 (SLC2A1), is a uniporter protein that in humans is encoded by the SLC2A1 gene. GLUT1 facilitates the transport of glucose across the plasma membranes of mammalian cells. This gene encodes a major glucose transporter in the mammalian blood-brain barrier.

In some aspects, a targeting moiety comprises galactose, e.g., D-galactose, which can bind to GLUT1 transporter to cross BBB. In some aspects, a targeting moiety comprises glutamic acid, which can bind to acetylcholinesterase inhibitor (AChEI) and/or EAATs inhibitors and cross BBB. Acetylcholinesterase is the enzyme that is the primary member of the cholinesterase enzyme family. An acetylcholinesterase inhibitor (AChEI) is the inhibitor that inhibits acetylcholinesterase from breaking down acetylcholine into choline and acetate, thereby increasing both the level and duration of action of the neurotransmitter acetylcholine in the central nervous system, autonomic ganglia and neuromuscular junctions, which are rich in acetylcholine receptors. Acetylcholinesterase inhibitors are one of two types of cholinesterase inhibitors; the other being butyryl-cholinesterase inhibitors.

In some aspects, the tissue targeted by a targeting moiety is a skeletal muscle. In some aspects, the targeting moiety targeting skeletal muscle is capable being transported by Large-neutral Amino Acid Transporter 1 (LAT1).

It is expressed in numerous cell types including T-cells, cancer cells and brain endothelial cells. LAT1 is consistently expressed at high levels in brain microvessel endothelial cells. Being a solute carrier located primarily in the BBB, targeting the extracellular vesicles of the present disclosure to LAT1 allows delivery through the BBB. In some aspects, the targeting moiety targeting an extracellular vesicle of the present disclosure to the LAT1 transporter is an amino acid, e.g., a branched-chain or aromatic amino acid. In some aspects, the amino acid is valine, leucine, and/or isoleucine. In some aspects, the amino acid is tryptophan and/or tyrosine. In some aspects, the amino acid is tryptophan. In other aspects, the amino acid is tyrosine.

In some aspects, the targeting moiety is a LAT1 ligand selected from tryptophan, tyrosine, phenylalanine, tryptophan, methionine, thyroxine, melphalan, L-DOPA, gabapentin, 3,5-I-diiodotyrosine, 3-iodo-I-tyrosine, fenclonine, acivicin, leucine, BCH, methionine, histidine, valine, or any combination thereof.

See Singh & Ecker (2018) “Insights into the Structure, Function, and Ligand Discovery of the Large Neutral Amino Acid Transporter 1, LAT1,” Int. J. Mol. Sci. 19:1278; Geier et al. (2013) “Structure-based ligand discovery for the Large-neutral Amino Acid Transporter 1, LAT-1,” Proc. Natl. Acad. Sci. USA 110:5480-85; and Chien et al. (2018) “Reevaluating the Substrate Specificity of the L-type Amino Acid Transporter (LAT1),” J. Med. Chem. 61:7358-73, which are herein incorporated by reference in their entireties.

In some aspects, a targeting moiety comprises tyrosine, which can bind to LAT1 and cross BBB. In some aspects, a targeting moiety comprises lysine, which can bind to LAT1 and cross BBB. In some aspects, a targeting moiety comprises glutamine, which can bind to LAT1 and cross BBB. In some aspects, a targeting moiety comprises phenylalanine, which can bind to GABA receptors, LAT1, CNS reverse transcriptase inhibitors, and/or dopamine (DA) receptors and cross BBB. Dopamine receptors are a class of G protein-coupled receptors that are prominent in the vertebrate central nervous system (CNS). Dopamine receptors activate different effectors through not only G-protein coupling, but also signaling through different protein (dopamine receptor-interacting proteins) interactions. The neurotransmitter dopamine is the primary endogenous ligand for dopamine receptors.

Dopamine receptors are implicated in many neurological processes, including motivation, pleasure, cognition, memory, learning, and fine motor control, as well as modulation of neuroendocrine signaling. Abnormal dopamine receptor signaling and dopaminergic nerve function is implicated in several neuropsychiatric disorders. Thus, dopamine receptors are common neurologic drug targets; antipsychotics are often dopamine receptor antagonists while psychostimulants are typically indirect agonists of dopamine receptors.

In some aspects, a targeting moiety comprises valine, which can bind to CNS reverse transcriptase inhibitors and cross BBB. In some aspects, a targeting moiety comprises tryptophan, which can bind to GABA receptors and/or CNS reverse transcriptase inhibitors and cross BBB. In some aspects, a targeting moiety comprises leucine, which can bind to GABA receptors and/or CNS reverse transcriptase inhibitors and cross BBB. In some aspects, a targeting moiety comprises methionine, which can bind to GABA receptors and/or CNS reverse transcriptase inhibitors and cross BBB. In some aspects, a targeting moiety comprises histidine, which can bind to GABA receptors and cross BBB. In some aspects, a targeting moiety comprises isoleucine, which can bind to CNS reverse transcriptase inhibitors and cross BBB. In some aspects, a targeting moiety comprises Glutathione, which can bind to GSH transporter and cross BBB. In some aspects, a targeting moiety comprises Glutathione-Met, which can bind to GSH transporter and cross BBB. In some aspects, a targeting moiety comprises Urea/Thiourea, which can bind to Nitric oxide synthase (NOS) and bind to BBB. In some aspects, a targeting moiety comprises NAD+/NADH, which is capable of crossing BBB by REDOX mechanism. In some aspects, a targeting moiety comprises purine and can cross BBB. Additional examples of targeting moieties for CNS targeting are shown in Sutera et al. (2016): Small endogenous molecules as moiety to improve targeting of CNS drugs, Expert Opinion on Drug Delivery, DOI: 10.1080/17425247.2016.1208651, which is incorporated herein by reference in its entirety.

In some aspects, the tissue targeted by a targeting moiety is a skeletal muscle. In some aspects, the targeting moiety targeting skeletal muscle is capable being transported by Large-neutral Amino Acid Transporter 1 (LAT1).

It is expressed in numerous cell types including T-cells, cancer cells and brain endothelial cells. LAT1 is consistently expressed at high levels in brain microvessel endothelial cells. Being a solute carrier located primarily in the BBB, targeting the extracellular vesicles of the present disclosure to LAT1 allows delivery through the BBB. In some aspects, the targeting moiety targeting an extracellular vesicle of the present disclosure to the LAT1 transporter is an amino acid, e.g., a branched-chain or aromatic amino acid. In some aspects, the amino acid is valine, leucine, and/or isoleucine. In some aspects, the amino acid is tryptophan and/or tyrosine. In some aspects, the amino acid is tryptophan. In other aspects, the amino acid is tyrosine.

In some aspects, the targeting moiety is a LAT1 ligand selected from tryptophan, tyrosine, phenylalanine, tryptophan, methionine, thyroxine, melphalan, L-DOPA, gabapentin, 3,5-I-diiodotyrosine, 3-iodo-I-tyrosine, fenclonine, acivicin, leucine, BCH, methionine, histidine, valine, or any combination thereof.

In some aspects, the LAT1 ligand is [1] 1-Phenylalanine, [2] o-Sarcolysin, [3] m-Sarcolysin. [4] Melphalan. [5] 2-Amino-2-norbornanecarboxylic acid (BCH). [6] (±)-2-Amino-1,2,3,4-tetrahydro-2-naphthoic acid, [7] dl-2-NAM-5, [8] dl-2-NAM-6, [9] dl-2-NAM-7, [10] dl-2-NAM-8, [11] dl-dechlorinated-NAM, [12] dl-1-NAM-7, [13] (±)-2-Aminoindane-2 carboxylic acid, [14] (±)-2-Aminobenzo-bicyclo-[2.2.1]heptane-2′-exo-carboxylic acid, [15] (±)-2-amino-(bis-2-chloroethyl)-5-aminoindane-2-carboxylic acid, [16] (±)-2-endo-amino-bis(2-chloroethyl)-7′-aminobenzobicyclo[2.2.1]heptane-2-exo-carboxylic acid, [17] 1-6-diazo-5-oxo-norleucine (1-DON), [18] Acivicin, [19] Azaserine, [20] Buthionine Sulfoximine (BSO), [21] 1-1-naphthylalanine, [22] o-benzyl-1-tyrosine, [23]1-2-amino-nonanoic acid, [24]1-Tyrosine, [25] α-methyltyrosine, [26]1-DOPA, [27] α-methyldopa, [28] 3-o-methyldopa, [29] Droxidopa, [30] Carbidopa, [31] Dopamine, [32] Tyramine, [33] α-methylphenylalanine, [34] N-methylphenylalanine, [35] Phenylalanine methyl ester, [36] Gabapentin, [37] 3,3′-diiodothyronine, [38] 1-T3, [39] 3′,5′,3-triiodothyronine (r 1-T3), or [40], 1-T4, or any combination thereof, as shown below.

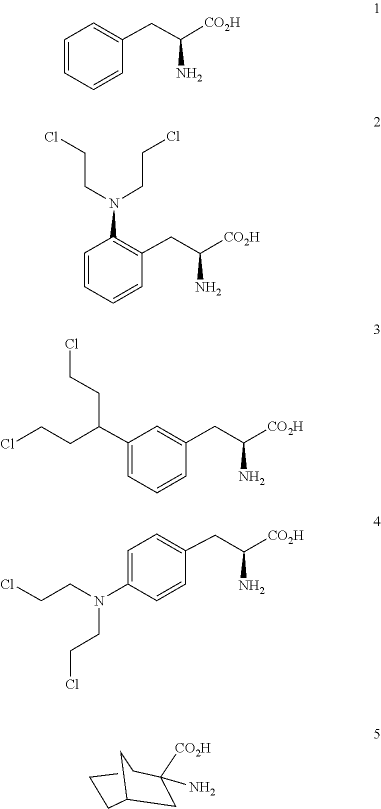

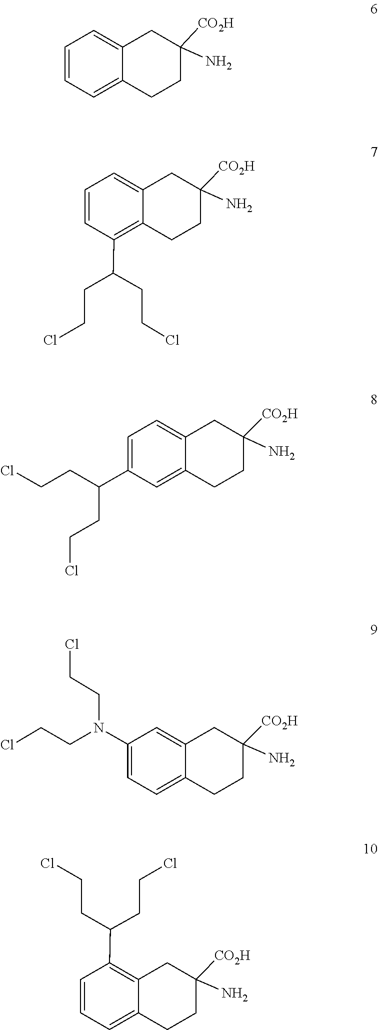 [SMILES: NC1(C(=O)O)CCc2ccccc2C1 ; NC1(C(=O)O)CCc2c(cccc2C(CCCl)CCCl)C1 ; NC1(C(=O)O)CCc2cc(C(CCCl)CCCl)ccc2C1 ; NC1(C(=O)O)CCc2ccc(N(CCCl)CCCl)cc2C1 ; NC1(C(=O)O)CCc2cccc(C(CCCl)CCCl)c2C1]

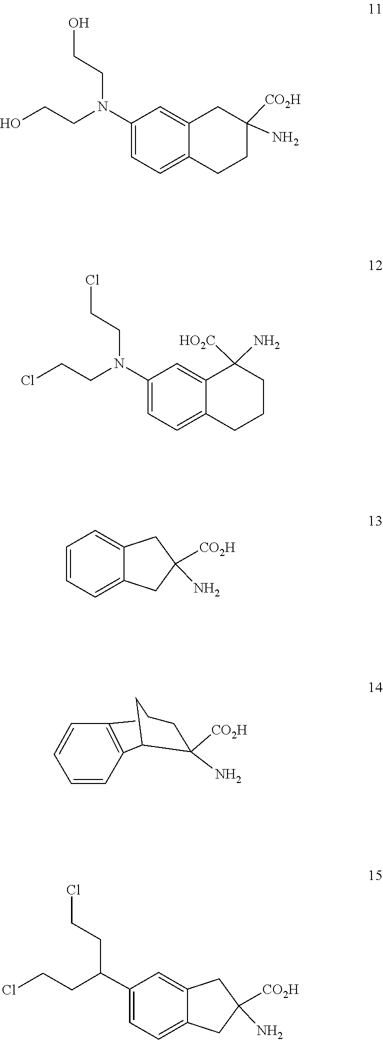 [SMILES: NC1(C(=O)O)CCc2ccc(N(CCO)CCO)cc2C1 ; NC1(C(=O)O)CCCc2ccc(N(CCCl)CCCl)cc21 ; NC1(C(=O)O)Cc2ccccc2C1 ; Cc1ccccc1C1CCCC1(N)C(=O)O ; NC1(C(=O)O)Cc2ccc(C(CCCl)CCCl)cc2C1]

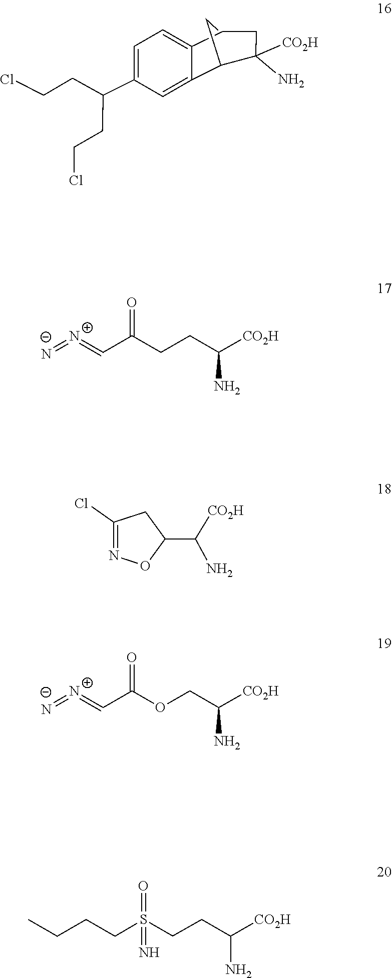 [SMILES: CCC1c2cc(C(CCCl)CCCl)ccc2CCC1(N)C(=O)O ; [N-]=[N+]=CC(=O)CC[C@H](N)C(=O)O ; NC(C(=O)O)C1CC(Cl)=NO1 ; [N-]=[N+]=CC(=O)OC[C@H](N)C(=O)O ; CCCCS(=N)(=O)CCC(N)C(=O)O]

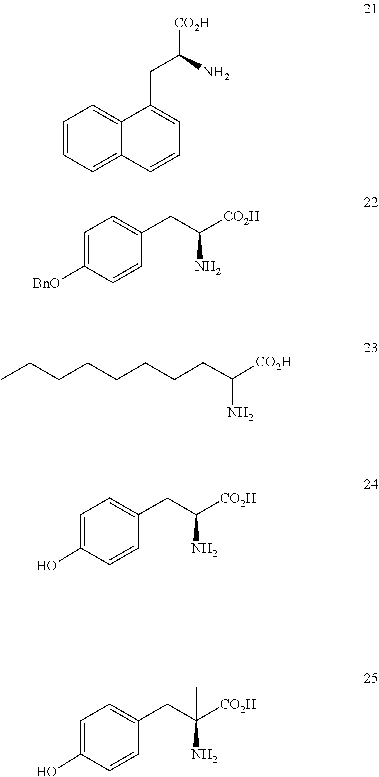 [SMILES: N[C@@H](Cc1cccc2ccccc12)C(=O)O ; N[C@@H](Cc1ccc(OCc2ccccc2)cc1)C(=O)O ; CCCCCCCCC(N)C(=O)O ; N[C@@H](Cc1ccc(O)cc1)C(=O)O ; C[C@@](N)(Cc1ccc(O)cc1)C(=O)O]

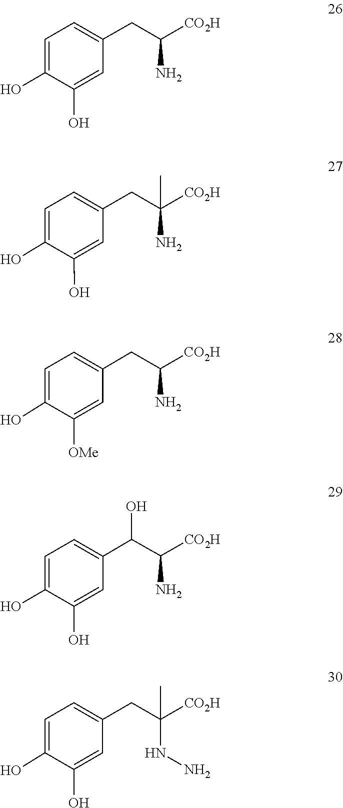 [SMILES: N[C@@H](Cc1ccc(O)c(O)c1)C(=O)O ; C[C@@](N)(Cc1ccc(O)c(O)c1)C(=O)O ; COc1cc(C[C@H](N)C(=O)O)ccc1O ; N[C@H](C(=O)O)C(O)c1ccc(O)c(O)c1 ; CC(Cc1ccc(O)c(O)c1)(NN)C(=O)O]

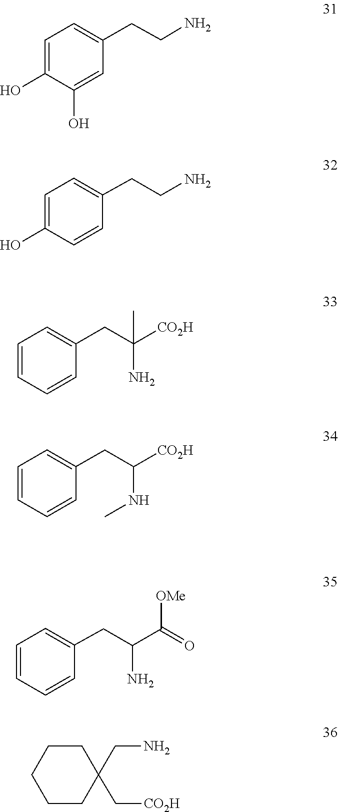

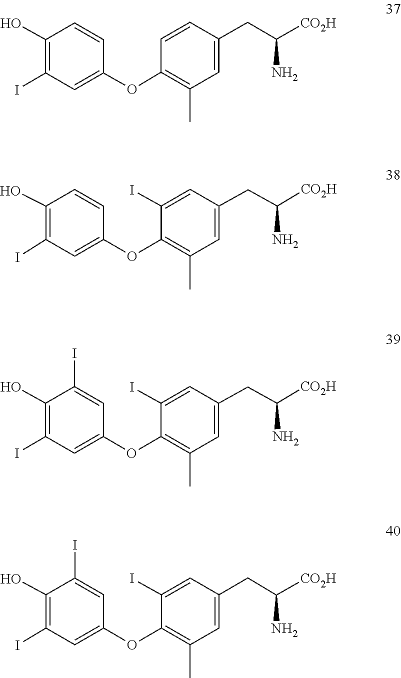 [SMILES: Cc1cc(C[C@H](N)C(=O)O)ccc1Oc1ccc(O)c(I)c1 ; Cc1cc(C[C@H](N)C(=O)O)cc(I)c1Oc1ccc(O)c(I)c1 ; Cc1cc(C[C@H](N)C(=O)O)cc(I)c1Oc1cc(I)c(O)c(I)c1 ; Cc1cc(C[C@H](N)C(=O)O)cc(I)c1Oc1cc(I)c(O)c(I)c1]

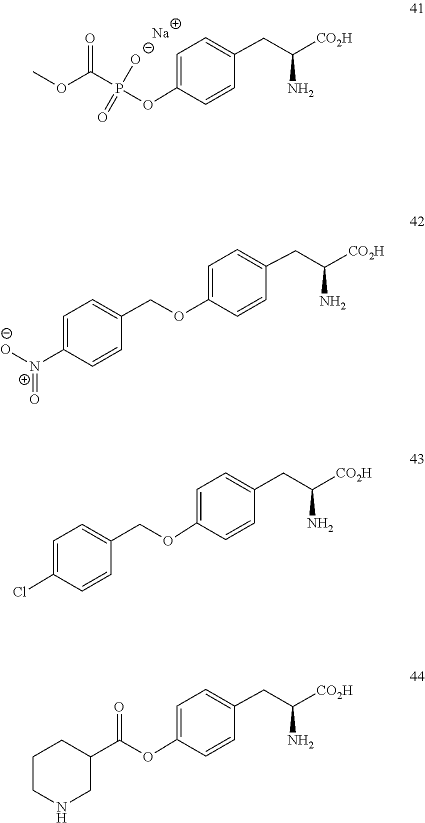

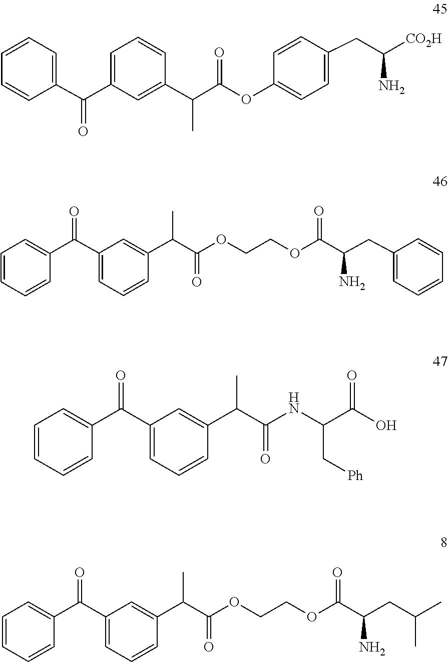 [SMILES: CC(C(=O)Oc1ccc(C[C@H](N)C(=O)O)cc1)c1cccc(C(=O)c2ccccc2)c1 ; CC(C(=O)OCCOC(=O)[C@H](N)Cc1ccccc1)c1cccc(C(=O)c2ccccc2)c1 ; CC(C(=O)NC(Cc1ccccc1)C(=O)O)c1cccc(C(=O)c2ccccc2)c1 ; CC(C)C[C@@H](N)C(=O)OCCOC(=O)C(C)c1cccc(C(=O)c2ccccc2)c1]

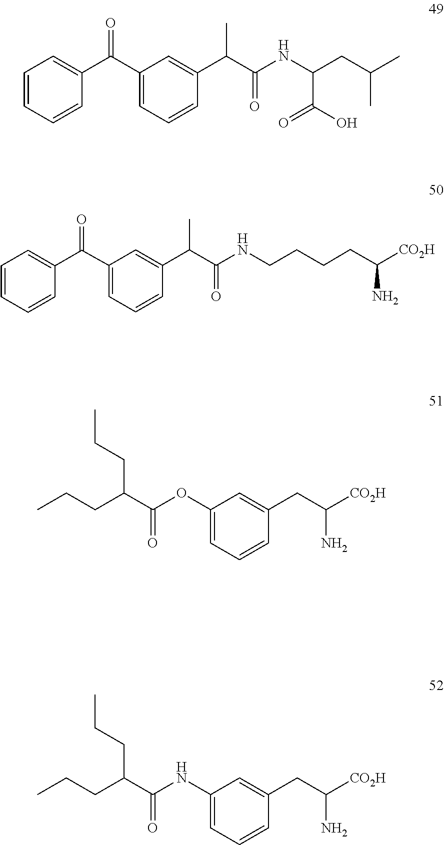

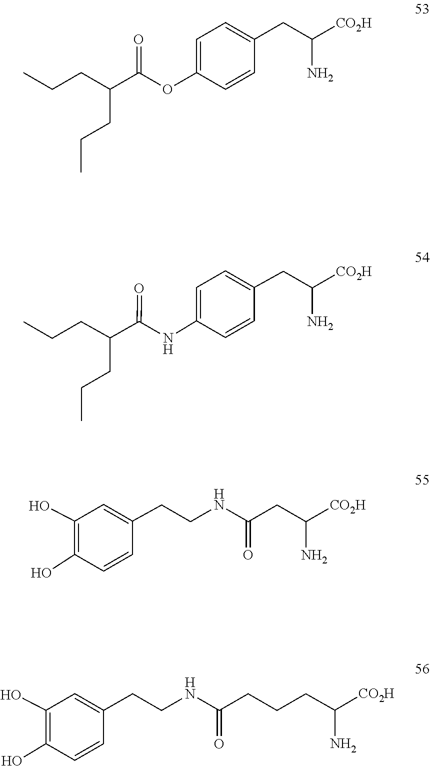 [SMILES: CCCC(CCC)C(=O)Oc1ccc(CC(N)C(=O)O)cc1 ; CCCC(CCC)C(=O)Nc1ccc(CC(N)C(=O)O)cc1 ; NC(CC(=O)NCCc1ccc(O)c(O)c1)C(=O)O ; NC(CCCC(=O)NCCc1ccc(O)c(O)c1)C(=O)O]

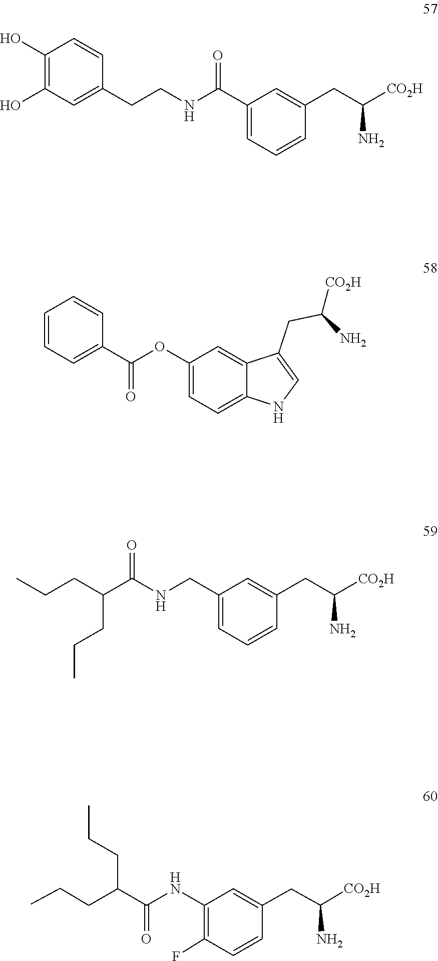 [SMILES: N[C@@H](Cc1cccc(C(=O)NCCc2ccc(O)c(O)c2)c1)C(=O)O ; N[C@@H](Cc1c[nH]c2ccc(OC(=O)c3ccccc3)cc12)C(=O)O ; CCCC(CCC)C(=O)NCc1cccc(C[C@H](N)C(=O)O)c1 ; CCCC(CCC)C(=O)Nc1cc(C[C@H](N)C(=O)O)ccc1F]

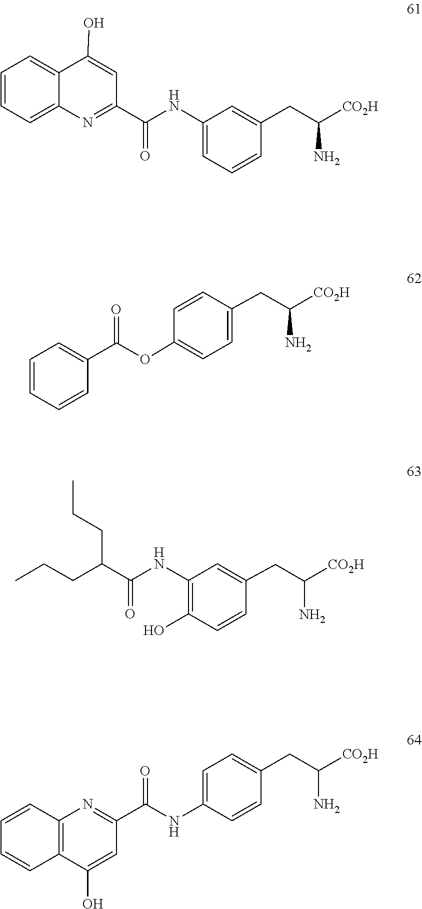 [SMILES: N[C@@H](Cc1cccc(NC(=O)c2cc(O)c3ccccc3n2)c1)C(=O)O ; N[C@@H](Cc1ccc(OC(=O)c2ccccc2)cc1)C(=O)O ; CCCC(CCC)C(=O)Nc1cc(CC(N)C(=O)O)ccc1O ; NC(Cc1ccc(NC(=O)c2cc(O)c3ccccc3n2)cc1)C(=O)O]

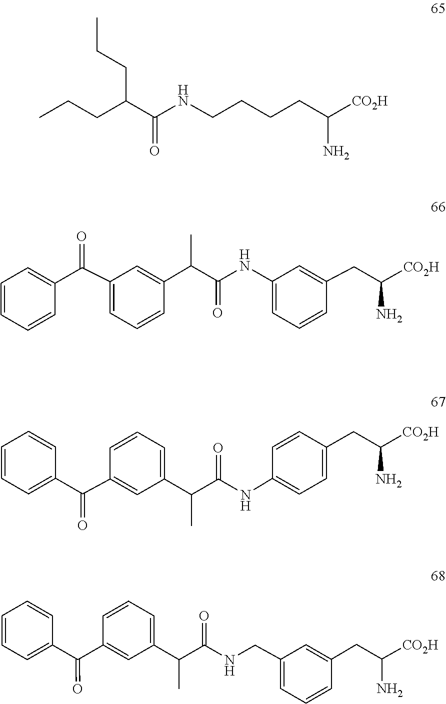 [SMILES: CCCC(CCC)C(=O)NCCCCC(N)C(=O)O ; CC(C(=O)Nc1cccc(C[C@H](N)C(=O)O)c1)c1cccc(C(=O)c2ccccc2)c1 ; CC(C(=O)Nc1ccc(C[C@H](N)C(=O)O)cc1)c1cccc(C(=O)c2ccccc2)c1 ; CC(C(=O)NCc1cccc(CC(N)C(=O)O)c1)c1cccc(C(=O)c2ccccc2)c1]

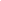

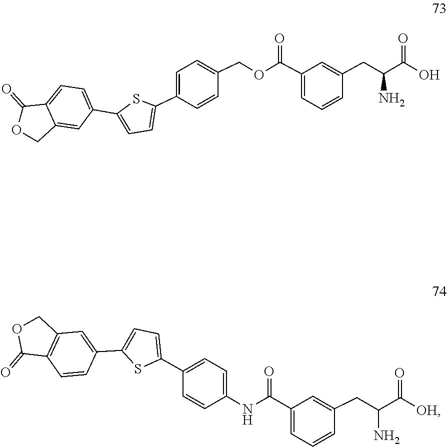 [SMILES: N[C@@H](Cc1cccc(C(=O)OCc2ccc(-c3ccc(-c4ccc5c(c4)COC5=O)s3)cc2)c1)C(=O)O ; NC(Cc1cccc(C(=O)Nc2ccc(-c3ccc(-c4ccc5c(c4)COC5=O)s3)cc2)c1)C(=O)O]

or any combination thereof.

In some aspects, the LAT1 ligand is a LAT1-targeting prodrug shown below.

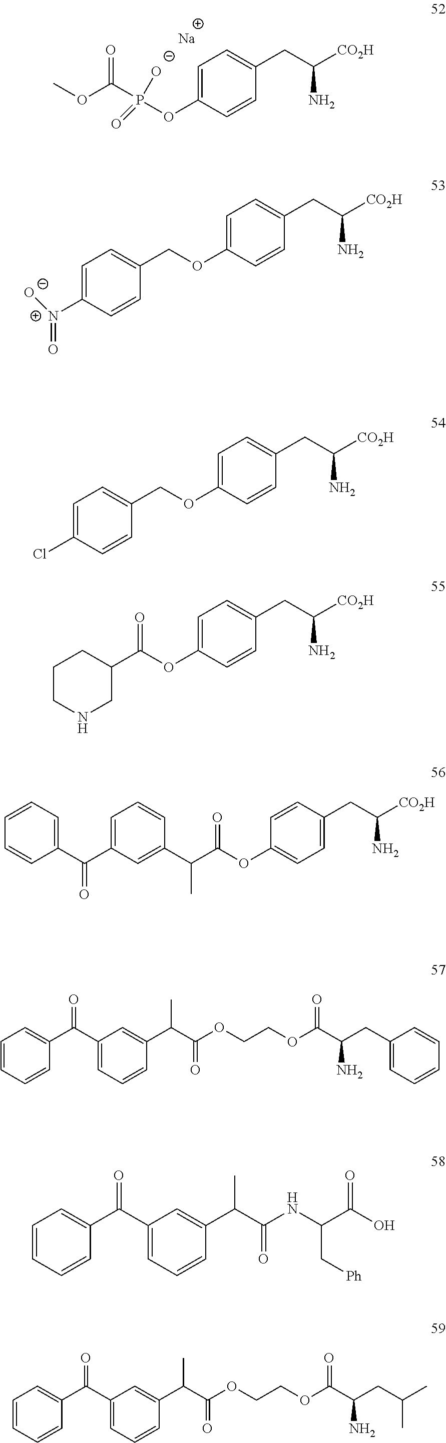

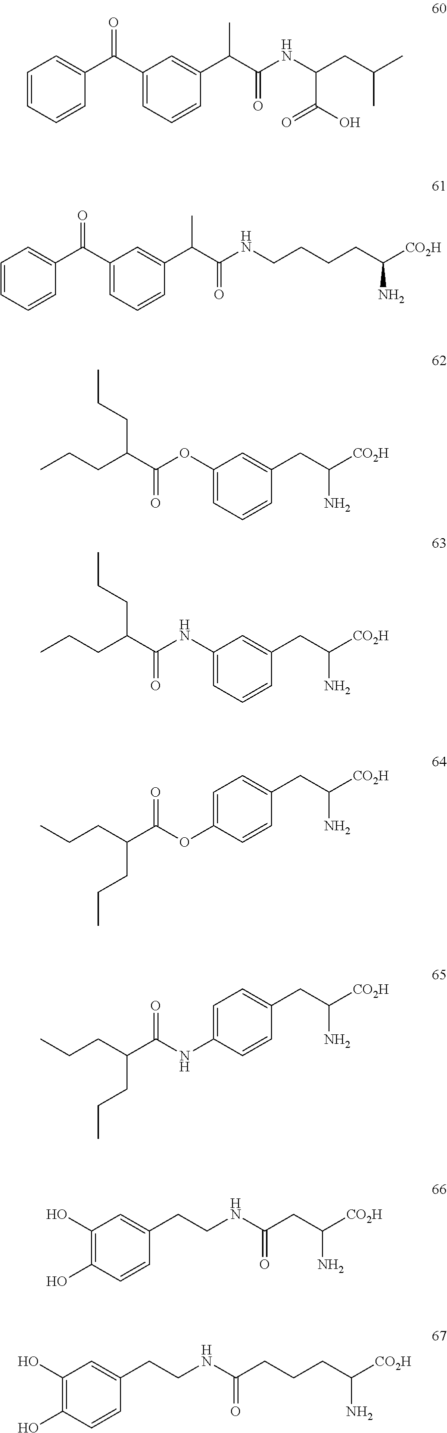

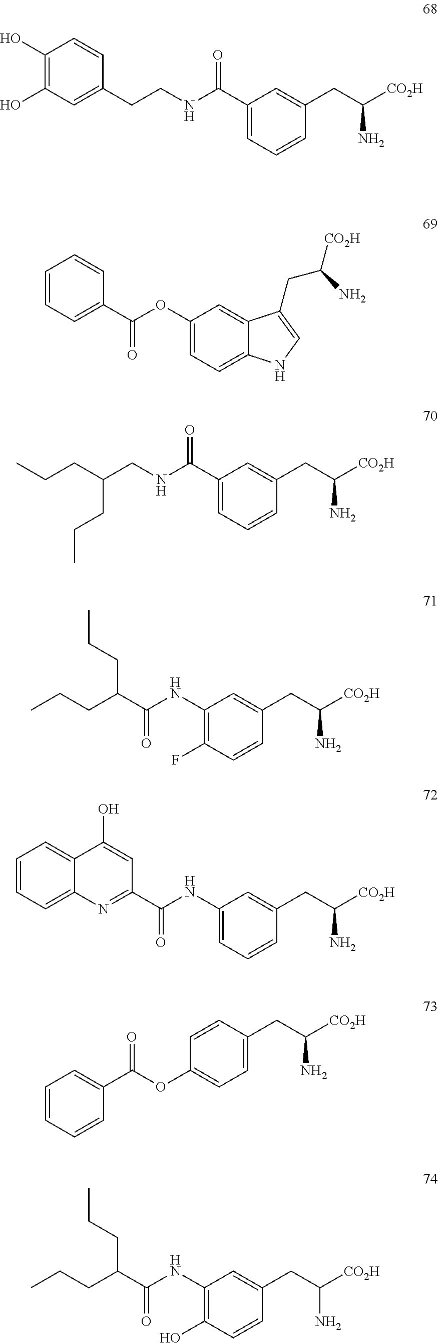

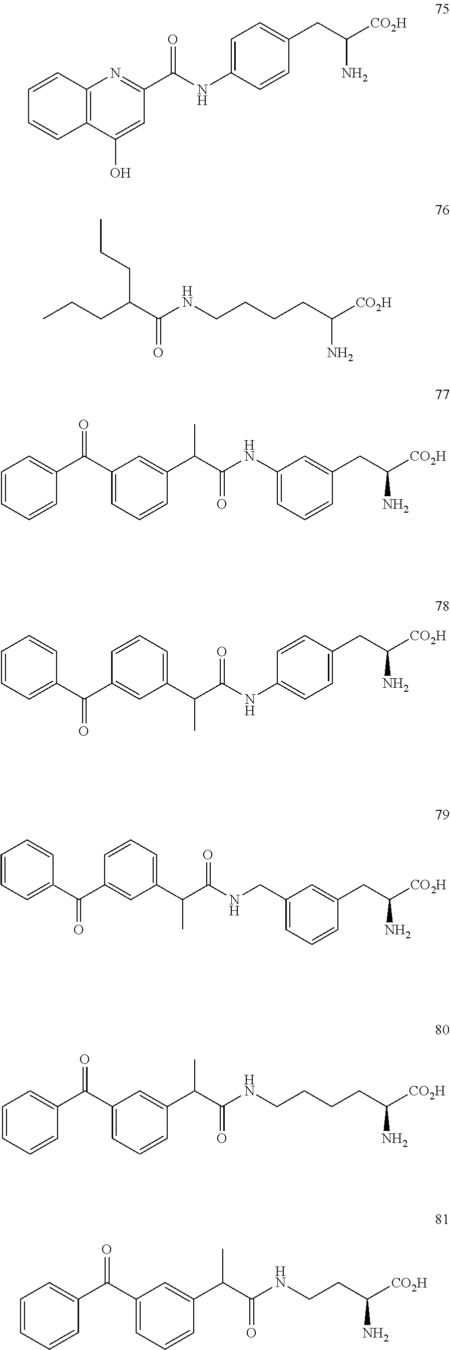

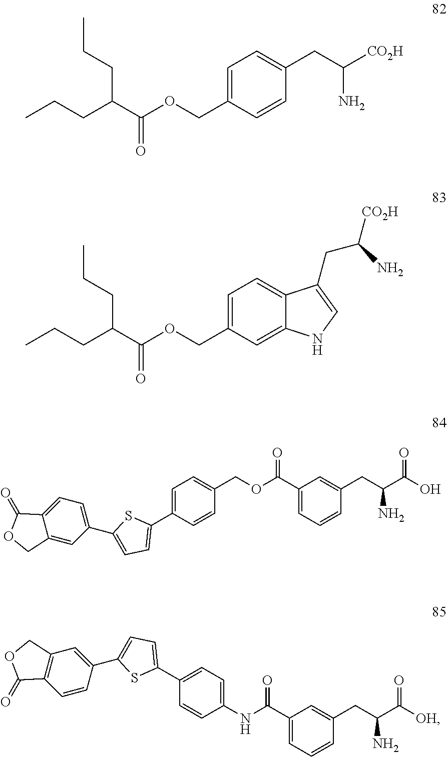 [SMILES: CCCC(CCC)C(=O)OCc1ccc(CC(N)C(=O)O)cc1 ; CCCC(CCC)C(=O)OCc1ccc2c(C[C@H](N)C(=O)O)c[nH]c2c1 ; N[C@@H](Cc1cccc(C(=O)OCc2ccc(-c3ccc(-c4ccc5c(c4)COC5=O)s3)cc2)c1)C(=O)O ; N[C@@H](Cc1cccc(C(=O)Nc2ccc(-c3ccc(-c4ccc5c(c4)COC5=O)s3)cc2)c1)C(=O)O]

or any combination thereof.

See Singh & Ecker (2018) “Insights into the Structure, Function, and Ligand Discovery of the Large Neutral Amino Acid Transporter 1, LAT1,” Int. J. Mol. Sci. 19:1278; Geier et al. (2013) “Structure-based ligand discovery for the Large-neutral Amino Acid Transporter 1, LAT-1,” Proc. Natl. Acad. Sci. USA 110:5480-85; and Chien et al. (2018) “Reevaluating the Substrate Specificity of the L-type Amino Acid Transporter (LAT1),” J. Med. Chem. 61:7358-73, which are herein incorporated by reference in their entireties.

A ligand functions as a type of targeting moiety defined as a selectively bindable material that has a selective (or specific), affinity for another substance. The ligand is recognized and bound by a usually, but not necessarily, larger specific binding body or “binding partner,” or “receptor.” Examples of ligands suitable for targeting are antigens, haptens, biotin, biotin derivatives, lectins, galactosamine and fucosylamine moieties, receptors, substrates, coenzymes and cofactors among others.

When applied to the EV of the present disclosure a ligand includes an antigen or hapten that is capable of being bound by, or to, its corresponding antibody or fraction thereof. Also included are viral antigens or hemagglutinins and neuraminidases and nucleocapsids including those from any DNA and RNA viruses, AIDS, HIV and hepatitis viruses, adenoviruses, alphaviruses, arenaviruses, coronaviruses, flaviviruses, herpesviruses, myxoviruses, oncornaviruses, papovaviruses, paramyxoviruses, parvoviruses, picornaviruses, poxviruses, reoviruses, rhabdoviruses, rhinoviruses, togaviruses and viroids; any bacterial antigens including those of gram-negative and gram-positive bacteria, Acinetobacter, Achromobacter, Bacteroides, Clostridium, Chlamydia, enterobacteria, Haemophilus, Lactobacillus, Neisseria, Staphyloccus, or Streptoccocus; any fungal antigens including those of Aspergillus, Candida, Coccidiodes, mycoses, phycomycetes, and yeasts; any mycoplasma antigens; any rickettsial antigens; any protozoan antigens; any parasite antigens; any human antigens including those of blood cells, virus infected cells, genetic markers, heart diseases, oncoproteins, plasma proteins, complement factors, rheumatoid factors. Included are cancer and tumor antigens such as alpha-fetoproteins, prostate specific antigen (PSA) and CEA, cancer markers and oncoproteins, among others.

Other substances that can function as ligands for targeting an EV of the present disclosure are certain vitamins (i.e. folic acid, B12), steroids, prostaglandins, carbohydrates, lipids, antibiotics, drugs, digoxins, pesticides, narcotics, neuro-transmitters, and substances used or modified such that they function as ligands.

In some aspects, the targeting moiety comprises a protein or protein fragment (e.g., hormones, toxins), and synthetic or natural polypeptides with cell affinity. Ligands also include various substances with selective affinity for ligators that are produced through recombinant DNA, genetic and molecular engineering. Except when stated otherwise, ligands of the instant disclosure also include ligands as defined in U.S. Pat. No. 3,817,837, which is herein incorporated by reference in its entirety.

### IIB. Linkers

As described above, a targeting moiety and/or a biologically active molecule disclosed herein can comprise, one or more linkers. As used herein, the term “linker” refers to a peptide or polypeptide sequence (e.g., a synthetic peptide or polypeptide sequence), or a non-peptide linker for which its main function is to connect two moieties in a cationic carrier unit disclosed herein. In some aspects, EVs of the present disclosure can comprise at least one linker connecting a tissue-specific targeting moiety (TM) with a surface of the EV, at least one linker connecting a biologically active molecule with the exterior surface or interior source of the EVs. In some aspects, two or more linkers can be linked in tandem.

When multiple linkers are present on the EVs disclosed herein, each of the linkers can be the same or different. Generally, linkers provide flexibility to the targeting moiety and/or biologically active molecules. Linkers are not typically cleaved; however, in certain aspects, such cleavage can be desirable. Accordingly, in some aspects a linker can comprise one or more protease-cleavable sites, which can be located within the sequence of the linker or flanking the linker at either end of the linker sequence.

In one aspect, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker can comprise at least about 110, at least about 120, at least about 130, at least about 140, at least about 150, at least about 160, at least about 170, at least about 180, at least about 190, or at least about 200 amino acids.

In other aspects, the peptide linker can comprise at least about 200, at least about 250, at least about 300, at least about 350, at least about 400, at least about 450, at least about 500, at least 550, at least about 600, at least about 650, at least about 700, at least about 750, at least about 800, at least about 850, at least about 900, at least about 950, or at least about 1,000 amino acids.

The peptide linker can comprise between 1 and about 5 amino acids, between 1 and about 10 amino acids, between 1 and about 20 amino acids, between about 10 and about 50 amino acids, between about 50 and about 100 amino acids, between about 100 and about 200 amino acids, between about 200 and about 300 amino acids, between about 300 and about 400 amino acids, between about 400 and about 500 amino acids, between about 500 and about 600 amino acids, between about 600 and about 700 amino acids, between about 700 and about 800 amino acids, between about 800 and about 900 amino acids, or between about 900 and about 1000 amino acids.

Examples of peptide linkers are well known in the art. In some aspects, the linker is a glycine/serine linker. In some aspects, the peptide linker is glycine/serine linker according to the formula [(Gly)n-Ser]m (SEQ ID NO: 57) where n is any integer from 1 to 100 and m is any integer from 1 to 100. In other aspects the glycine/serine linker is according to the formula [(Gly)x-Sery]z (SEQ ID NO: 1) wherein x in an integer from 1 to 4, y is 0 or 1, and z is an integers from 1 to 50. In one aspect, the peptide linker comprises the sequence Gn (SEQ ID NO: 58), where n can be an integer from 1 to 100. In a specific aspect, the sequence of the peptide linker is GGGG (SEQ ID NO: 59).

In some aspects, the peptide linker can comprise the sequence (Gly Ala)n (SEQ ID NO: 60), wherein n is an integer between 1 and 100. In other aspects, the peptide linker can comprise the sequence (GlyGlySer)n (SEQ ID NO: 61), wherein n is an integer between 1 and 100.

In other aspects, the peptide linker comprises the sequence (GGGS)n (SEQ ID NO: 62). In still other aspects, the peptide linker comprises the sequence (GGS)n(GGGGS)n (SEQ ID NO: 63). In these instances, n can be an integer from 1-100. In other instances, n can be an integer from one to 20, i.e., 1, 2, 3, 4, 5, 6, 7, 8, 9, 10, 11, 12, 13, 14, 15, 16, 17, 18, 19, or 20.

Examples of linkers include, but are not limited to, GGG, SGGSGGS (SEQ ID NO: 64), GGSGGSGGSGGSGGG (SEQ ID NO: 65), GGSGGSGGGGSGGGGS (SEQ ID NO: 66), GGSGGSGGSGGSGGSGGS (SEQ ID NO: 67), or GGGGSGGGGSGGGGS (SEQ ID NO: 68). In other aspects, the linker is a poly-G sequence (GGGG)n (SEQ ID NO: 69), where n can be an integer from 1-100.

In one aspect, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides that are modified forms of naturally occurring polypeptides (e.g., comprising a mutation such as an addition, substitution or deletion). In another aspect, the peptide linker can comprise non-naturally occurring amino acids. In another aspect, the peptide linker can comprise naturally occurring amino acids occurring in a linear sequence that does not occur in nature. In still another aspect, the peptide linker can comprise a naturally occurring polypeptide sequence.

In some aspects, the linker comprises a non-peptide linker. In other aspects, the linker consists of a non-peptide linker. In some aspects, the non-peptide linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl)butyrate (SMPB), N-succinimidyl(4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio)toluene (SMPT), etc. (see, e.g., U.S. Pat. No. 7,375,078).

Linkers can be introduced into polypeptide sequences using techniques known in the art (e.g., chemical conjugation, recombinant techniques, or peptide synthesis). Modifications can be confirmed by DNA sequence analysis. In some aspects, the linkers are introduced using recombinant techniques. In other aspects, the linkers are introduced using solid phase peptide synthesis. In some aspects, a EV disclosed herein can contain simultaneously one or more linkers that have been introduced using recombinant techniques and one or more linkers that have been introduced using solid phase peptide synthesis or methods of chemical conjugation known in the art. In some aspects, the linker comprises a cleavage site.

### IIC. Biologically Active Molecule

As used herein the term “biologically active molecule” refers to a payload and can be a non therapeutic agent or a therapeutic agent that can be interactive by itself or via an adapter with an EV of the present disclosure, and be included within the core of an EV or attached to the exterior surface of an EV of the present disclosure.

Other biologically active molecules are anti-viral drugs, nucleic acids and other anti-viral substances including those against any DNA and RNA viruses, AIDS, HIV and hepatitis viruses, adenoviruses, alphaviruses, arenaviruses, coronaviruses, flaviviruses, herpesviruses, myxoviruses, oncornaviruses, papovaviruses, paramyxoviruses, parvoviruses, picomaviruses, poxviruses, reoviruses, thabdoviruses, rhinoviruses, togaviruses and viriods; any anti-bacterial drugs, nucleic acids and other anti-bacterial substances including those against gram-negative and grampositive bacteria, Acinetobacter, Achromobacter, Bacteroides, Clostridium, Chlamydia, enterobacteria, Haemophilus, Lactobacillus, Neisseria, Staphyloccus, or Streptoccocus; any antifungal drugs, nucleic acids and other anti-fungal substances including those against Aspergillus, Candida, Coccidiodes, mycoses, phycomycetes, and yeasts; any drugs, nucleic acids and other substances against mycoplasma and rickettsia; any anti-protozoan drugs, nucleic acids and other substances; any anti-parasitic drugs, nucleic acids and other substances; any drugs, nucleic acids and other substances against heart diseases, tumors, and virus infected cells, among others.

### (a) Nucleic Acids

In some aspects, the biologically active molecule (payload) is a nucleic acid, e.g., an RNA or a DNA. Nucleic acid active agents suitable for delivery using the extracellular vesicles of the present disclosure include all types of RNA and all types of DNA, including also oligonucleotides such as probes and primers used in the polymerase chain reaction (PCR), hybridizations, or DNA sequencing. In some aspects, the nucleic acid comprises mRNA, miRNA, miRNA sponge, tough decoy miRNA (TD), antimir (antagomir), small RNA, rRNA, siRNA, shRNA, gDNA, cDNA, pDNA, PNA, BNA, antisense oligonucleotide (ASO), aptamer, cyclic dinucleotide, or any combination thereof.

In some aspects, the biologically active molecule (payload) comprises a short interfering RNA (siRNA), which is a double-stranded RNA that can induce sequence-specific post-transcriptional gene silencing, thereby decreasing or even inhibiting gene expression. For example, siRNAs can trigger the specific degradation of homologous RNA molecules, such as mRNAs, within the region of sequence identity between both the siRNA and the target RNA. Non-limiting exemplary siRNAs are disclosed in WO 02/44321, which is incorporated by reference in its entirety. In some aspects, siRNA can be about 20-27 base pairs in length.

In some aspects, the siRNA can be chemically modified. In some aspects, the siRNA can be conjugated to cholesterol. In some aspects, the cholesterol can be conjugated to a 3′ end of a sense or antisense strand of the siRNA. In some aspects, the cholesterol can be conjugated to a 5′ end of a sense or antisense strand of the siRNA. In some aspects, the cholesterol can be conjugated to both the 3′ and 5′ end of a sense or antisense strand of the siRNA.

As used herein, the number of charge in siRNA is the number of nucleotides −2.

In some aspects, the biologically active molecule (e.g., anionic payload) comprises a short hairpin RNAs (shRNAs). In some aspects, the biologically active molecule comprises an miRNA or a miRNA inhibitor (antimiR). In some aspects, the biologically active molecule (e.g., anionic payload) can be 10-30 nucleotides in length, for example from 14-25 nucleotides in length. In some aspects, the biologically active molecule (e.g. anionic payload) has a length of 16-30 nucleotides, 18-25 nucleotides, particularly 18, 19, 20, 21, 22, 23, 24, or 25 nucleotides. In some aspects, the biologically active molecule (e.g., anionic payload) comprises a nucleotide sequence having less than 200 nucleotides in length. In some aspects, the anionic payload comprises a nucleotide sequence having less than about 150, about 140, about 130, about 120, about 110, about 100, about 90, about 80, about 70, about 60, about 50, about 40, about 30, about 25, about 24, about 23, about 22, about 21, about 20, about 19, about 18, about 17, about 16, about 15, about 14, about 13, about 12, about 11, or about 10 nucleotides in length. In some aspects, the anionic payload comprises a nucleotide sequence having from about 30 to about 10, from about 25 to about 11, from about 30 to about 15, from about 25 to about 15, from about 24 to about 15, or from about 23 to about 15 nucleotides in length. In some aspects, the anionic payload comprises a nucleotide sequence having about 30, about 29, about 28, about 27, about 26, about 25, about 24, about 23, about 22, about 21, about 20, about 19, about 18, about 17, about 16, about 15, about 14, or about 13 nucleotides in length. In some aspects, the anionic payload comprises a nucleotide sequence having about 22 nucleotides in length.

Sequences for miRNAs are available publicly, for example, through the miRBase registry (Griffiths-Jones, et al., Nucleic Acids Res., 36(Database Issue):D154-D158 (2008); Griffiths-Jones, et al., Nucleic Acids Res., 36(Database Issue): D140-D144 (2008); Griffiths-Jones, et al., Nucleic Acids Res., 36(Database Issue):D109-D111 (2008)) and other publically accessible databases.

In some aspects, the miRNA inhibitors are oligomers or polymers of ribonucleic acid (RNA) or deoxyribonucleic acid (DNA) or modifications thereof. In some aspects, the miRNA antagonists are antimir. Antimirs are a specific class of miRNA inhibitors that are described, for example, in US2007/0213292 to Stoffel et al. Antimirs are RNA-like oligonucleotides that contain various modifications for RNase protection and pharmacologic properties such as enhanced tissue and cellular uptake. Antimirs differ from normal RNA by having complete 2′-O-methylation of sugar, phosphorothioate backbone and a cholesterol-moiety at 3′-end.

Non-limiting examples of antimirs and other miRNA inhibitors are described in WO2009/020771, WO2008/091703, WO2008/046911, WO2008/074328, WO2007/090073, WO2007/027775, WO2007/027894, WO2007/021896, WO2006/093526, WO2006/112872, WO2007/112753, WO2007/112754, WO2005/023986, or WO2005/013901, all of which are hereby incorporated by reference.

In some aspects, the anionic payload comprises mRNA, miRNA, miRNA sponge, tough decoy miRNA, antimir, small RNA, rRNA, siRNA, shRNA, gDNA, cDNA, pDNA, PNA, BNA, antisense oligonucleotide (ASO), aptamer, cyclic dinucleotide, or any combination thereof. In some aspects, the anionic payload is an siRNA. In some aspects, the anionic payload is mRNA. In some aspects, the anionic payload is a PNA.

In some aspects, the nucleic acids are phosphodiester antisense oligonucleotides, and any oligonucleotides where the sugar-phosphate “backbone” has been derivatized or replaced with “backbone analogues” such as with phosphorothioate, phosphorodithioate, phosphoroamidate, alkyl phosphotriester, or methylphosphonate linkages. In some aspects, the nucleic acids active agents are antisense oligonucleotides, and any oligonucleotides or oligodeoxynucleotides with non-phosphorous backbone analogues such as sulfamate, 3′-thioformacetal, methylene(methylimino) (MMI), 3′-N-carbamate, or morpholino carbamate.

In some aspects, the biologically active molecule (payload) is an antimir. As used herein, the terms “antimir,” “anti microRNA,” “anti miRNA,” and variants thereof refer to molecules (e.g., synthetically generated molecules) that are used to neutralize microRNA (miRNA) function in cells for desired responses. miRNA are complementary sequences (approx. 20-22 bp) to mRNA that are involved in the cleavage of RNA or the suppression of the translation. By controlling the miRNA that regulate mRNAs in cells, antimirs (also called anti-miRNA oligonucleotides, AMOs, or antagomirs) can be used as further regulation as well as for therapeutic for certain cellular disorders. This regulation can occur through a steric blocking mechanism as well as hybridization to miRNA.

These interactions within the body between antimirs and a miRNA can be for therapeutics in disorders in which over/under expression occurs or aberrations in miRNA lead to coding issues. Some of the miRNA linked disorders that are encountered in the humans include cancers, muscular diseases, autoimmune disorders, and viruses.

Various components of antimirs can be manipulated to affect the binding affinity and potency of the antimir. The 2′-sugar of the antimirs can be modified to be substituted with fluorine and various methyl groups, almost all with an increase in binding affinity. However, some of these modified 2′-sugar antimirs lead to negative effects on cell growth. Modifying the 5′-3′ phosphodiester backbone linkage to a phosphorothioate (P-S) backbone linkage is also known to have an effect on target affinity. Using the P-S mutation was shown to decrease the Tm of the oligonucleotide, which leads to a lower target affinity. A final requirement for antimirs is mismatch specificity and length restrictions. Due to miRNAs in the same families sharing “seed” (shared) sequences and differ by only a couple of additional nucleotides; one antimir can potentially target multiple miRNA sequences. One or more examples of antimirs or miRNA sequences are shown in the following table.

TABLE 1

| | | | | | Artificial |
|---|---|---|---|---|---|
| | | | | | miRNA |
| SEQ ID | | | Mature | SEQ ID | inhibitor |
| NO for | Target | miRNA | miRNA | NO for | sequence |
| miRNA | Score | Name | sequence | antimir | (antimir) |
| 50 | 95 | hsa-miR- | UUCCCUUU | 51 | AGGCAUAGGA |
| | | 204-5p | GUCAUCCU | | UGACAAAGGG |
| | | | AUGCCU | | AA |
| 52 | 89 | hsa-miR- | UAACAGUC | 53 | CGACCAUGGC |
| | | 132-3p | UACAGCCA | | UGUAGACUGU |
| | | | UGGUCG | | UA |

In some aspects, the payload is a polynucleotide comprising a nucleotide sequence having 5 to 30 nucleotides in length. In some aspects, the polynucleotide has 5, 6, 7, 8, 9, 10, 11, 12, 13, 14, 15, 16, 17, 18, 19, 20, 21, 22, 23, 24, 25, 26, 27, 28, 29, or 30 nucleotides in length. In some aspects, the nucleotide sequence has 15, 16, 17, 18, 19, 20, 21, 22, 23, 24, 25, or 26 nucleotides in length. In some aspects, the payload comprises a nucleotide sequence having less than 200 nucleotides in length. In some aspects, the payload comprises a nucleotide sequence having less than about 150, about 140, about 130, about 120, about 110, about 100, about 90, about 80, about 70, about 60, about 50, about 40, about 30, about 25, about 24, about 23, about 22, about 21, about 20, about 19, about 18, about 17, about 16, about 15, about 14, about 13, about 12, about 11, or about 10 nucleotides in length. In some aspects, the payload comprises a nucleotide sequence having from about 30 to about 10, from about 25 to about 11, from about 30 to about 15, from about 25 to about 15, from about 24 to about 15, or from about 23 to about 15 nucleotides in length. In some aspects, the payload comprises a nucleotide sequence having about 30, about 29, about 28, about 27, about 26, about 25, about 24, about 23, about 22, about 21, about 20, about 19, about 18, about 17, about 16, about 15, about 14, or about 13 nucleotides in length.

In some aspects, the payload (e.g., antimir) is a nucleotide sequence targeting hsa-miR-485, e.g., hsa-miR-485-3p. In some aspects, the hsa-miR-485-3p has the sequence GUCAUACACGGCUCUCCUCUCU (SEQ ID NO: 54). In some aspects, the payload (e.g., antimir) is a nucleotide sequence comprising, consisting essentially of, or consisting of AGAGAGGAGAGCCGUGUAUGAC (SEQ ID NO: 25), wherein U can be optionally T (SEQ ID NO: 55). In some aspects, the payload (e.g., antimir) is a nucleotide sequence comprising, consisting essentially of, or consisting of AGAGAGGAGAGCCGUGUAUGAC (SEQ ID NO: 25), wherein the nucleotide sequence has one mismatch, two mismatches, three mismatches, or four mismatches. In some aspects, the payload (e.g., antimir) is a nucleotide sequence comprising, consisting essentially of, or consisting of AGAGAGGAGAGCCGUGUAUGAC (SEQ ID NO: 25), wherein the nucleotide sequence has one or two mismatches. In other aspects, the payload (e.g., antimir) is a nucleotide sequence targeting the seed sequence of hsa-miR-485-3p (UCAUACA). In some aspects, the payload (e.g., antimir) is a nucleotide sequence comprising UCAUACA, wherein U can be optionally T (complement of the seed), wherein the nucleotide sequence is about 10 nucleotides to 30 nucleotides (e.g., 10 to 25, 10 to 24, 10 to 23, 10 to 22, 10 to 21, 10 to 20, 10 to 19, or 10 to 18) in length. In some aspects, the payload (e.g., antimir) is a nucleotide sequence comprising UGUAUGA, wherein U can be optionally T (complement of the seed), wherein the nucleotide sequence comprises one, two three, four, five, six, seven, eight, nine, ten, 11, 12, 13, 14, 15, 16, 17, 18, 19, or 20 nucleic acids at the 5′ terminus of the complement of the seed sequence and/or one, two three, four, five, six, seven, eight, nine, ten, 11, 12, 13, 14, 15, 16, 17, 18, 19, or 20 nucleic acids at the 3′ terminus of the complement of the seed sequence.

In some aspects, the payload is a nucleotide sequence selected from the group consisting of: 5′-UGUAUGA-3′, 5′-GUGUAUGA-3′, 5′-CGUGUAUGA-3′, 5′-CCGUGUAUGA-3′ (SEQ ID NO: 2), 5′-GCCGUGUAUGA-3′ (SEQ ID NO: 3), 5′-AGCCGUGUAUGA-3′ (SEQ ID NO: 4), 5′-GAGCCGUGUAUGA-3′ (SEQ ID NO: 5), 5′-AGAGCCGUGUAUGA-3′ (SEQ ID NO: 6), 5′-GAGAGCCGUGUAUGA-3′ (SEQ ID NO: 7), 5′-GGAGAGCCGUGUAUGA-3′ (SEQ ID NO: 8), 5′-AGGAGAGCCGUGUAUGA-3′ (SEQ ID NO: 9), 5′-GAGGAGAGCCGUGUAUGA-3′ (SEQ ID NO: 10), 5′-AGAGGAGAGCCGUGUAUGA-3′ (SEQ ID NO: 11), 5′-GAGAGGAGAGCCGUGUAUGA-3′ (SEQ ID NO: 12); 5′-UGUAUGAC-3′, 5′-GUGUAUGAC-3′, 5′-CGUGUAUGAC-3′ (SEQ ID NO: 13), 5′-CCGUGUAUGAC-3′ (SEQ ID NO: 14), 5′-GCCGUGUAUGAC-3′ (SEQ ID NO: 15), 5′-AGCCGUGUAUGAC-3′ (SEQ ID NO: 16), 5′-GAGCCGUGUAUGAC-3′ (SEQ ID NO: 17), 5′-AGAGCCGUGUAUGAC-3′ (SEQ ID NO: 18), 5′-GAGAGCCGUGUAUGAC-3′ (SEQ ID NO: 19), 5′-GGAGAGCCGUGUAUGAC-3′ (SEQ ID NO: 20), 5′-AGGAGAGCCGUGUAUGAC-3′ (SEQ ID NO: 21), 5′-GAGGAGAGCCGUGUAUGAC-3′ (SEQ ID NO: 22), 5′-AGAGGAGAGCCGUGUAUGAC-3′ (SEQ ID NO: 23), or 5′-GAGAGGAGAGCCGUGUAUGAC-3′ (SEQ ID NO: 24).

In some aspects, the payload is a nucleotide sequence comprising 5′-TGTATGA-3′, 5′-GTGTATGA-3′, 5′-CGTGTATGA-3′, 5′-CCGTGTATGA-3′ (SEQ ID NO: 26), 5′-GCCGTGTATGA-3′ (SEQ ID NO: 27), 5′-AGCCGTGTATGA-3′ (SEQ ID NO: 28), 5′-GAGCCGTGTATGA-3′ (SEQ ID NO: 29), 5′-AGAGCCGTGTATGA-3′ (SEQ ID NO: 30), 5′-GAGAGCCGTGTATGA-3′ (SEQ ID NO: 31), 5′-GGAGAGCCGTGTATGA-3′ (SEQ ID NO: 32), 5′-AGGAGAGCCGTGTATGA-3′ (SEQ ID NO: 33), 5′-GAGGAGAGCCGTGTATGA-3′ (SEQ ID NO: 34), 5′-AGAGGAGAGCCGTGTATGA-3′ (SEQ ID NO: 35), 5′-GAGAGGAGAGCCGTGTATGA-3′ (SEQ ID NO: 36); 5′-TGTATGAC-3′, 5′-GTGTATGAC-3′, 5′-CGTGTATGAC-3′ (SEQ ID NO: 37), 5′-CCGTGTATGAC-3′ (SEQ ID NO: 38), 5′-GCCGTGTATGAC-3′ (SEQ ID NO: 39), 5′-AGCCGTGTATGAC-3′ (SEQ ID NO: 40), 5′-GAGCCGTGTATGAC-3′ (SEQ ID NO: 41), 5′-AGAGCCGTGTATGAC-3′ (SEQ ID NO: 42), 5′-GAGAGCCGTGTATGAC-3′ (SEQ ID NO: 43), 5′-GGAGAGCCGTGTATGAC-3′ (SEQ ID NO: 44), 5′-AGGAGAGCCGTGTATGAC-3′ (SEQ ID NO: 45), 5′-GAGGAGAGCCGTGTATGAC-3′ (SEQ ID NO: 46), 5′-AGAGGAGAGCCGTGTATGAC-3′ (SEQ ID NO: 47), or 5′-GAGAGGAGAGCCGTGTATGAC-3′ (SEQ ID NO: 48).

In some aspects, the payload (e.g., antimir) is a nucleotide sequence targeting hsa-miR-204, e.g., hsa-miR-204-5p. The hsa-miR-204-5p is shown at TABLE 1 as UUCCCUUUGUCAUCCUAUGCCU (SEQ ID NO: 50). In some aspects, the payload (e.g., antimir) is a nucleotide sequence consisting of, comprising, essentially or consisting of AGGCAUAGGAUGACAAAGGGAA (SEQ ID NO: 51), wherein U can be optionally T (SEQ ID NO: 56). In some aspects, the payload (e.g., antimir) is a nucleotide sequence comprising, consisting essentially of, or consisting of AGGCAUAGGAUGACAAAGGGAA (SEQ ID NO: 51), wherein U can be optionally T (SEQ ID NO: 56) and wherein the nucleotide sequence has one mismatch, two mismatches, three mismatches, or four mismatches. In some aspects, the payload (e.g., antimir) is a nucleotide sequence comprising, consisting essentially of, or consisting of AGGCAUAGGAUGACAAAGGGAA (SEQ ID NO: 51), wherein U can be optionally T (SEQ ID NO: 56) and wherein the nucleotide sequence has one or two mismatches. In other aspects, the payload (e.g., antimir) is a nucleotide sequence targeting the seed sequence of hsa-miR-204-5p (UCCCUUU). In some aspects, the payload (e.g., antimir) is a nucleotide sequence comprising AAAGGGA (complement of the seed), wherein U can be optionally T and wherein the nucleotide sequence is about 10 nucleotides to 30 nucleotides (e.g., 10 to 25, 10 to 24, 10 to 23, 10 to 22, 10 to 21, 10 to 20, 10 to 19, or 10 to 18) in length. In some aspects, the payload (e.g., antimir) is a nucleotide sequence comprising AAAGGGA (complement to the seed), wherein the nucleotide sequence comprises one, two three, four, five, six, seven, eight, nine, ten, 11, 12, 13, 14, 15, 16, 17, 18, 19, or 20 nucleic acids at the 5′ terminus of the complement of the seed sequence and/or one, two three, four, five, six, seven, eight, nine, ten, 11, 12, 13, 14, 15, 16, 17, 18, 19, or 20 nucleic acids at the 3′ terminus of the complement of the seed sequence.

In some aspects, the biologically active molecule (payload) is a nucleic acid, e.g., an RNA or a DNA. Nucleic acid active agents suitable for delivery using the extracellular vesicles of the present disclosure include all types of RNA and all types of DNA. In some aspects, the nucleic acid comprises mRNA, miRNA sponge, tough decoy miRNA (TD), cDNA, pDNA, PNA, BNA, aptamer, or any combination thereof.

In some aspects, the biologically active molecule (e.g., anionic payload) comprises a nucleotide sequence having less than 4,000 nucleotides in length. In some aspects, the biologically active molecule (e.g., anionic payload) comprises a nucleotide sequence having less than about 3,500, less than about 3,000, less than about 2,500, less than about 2,000, less than about 1,500, less than about 1,000, less than about 900, less than about 800, less than about 700, less than about 600, less than about 500, less than about 400, less than about 200, or less than about 150 in length.

In some aspects, the nucleic acids are phosphodiester nucleotides, and any nucleotides where the sugar-phosphate “backbone” has been derivatized or replaced with “backbone analogues” such as with phosphorothioate, phosphorodithioate, phosphoroamidate, alkyl phosphotriester, or methylphosphonate linkages. In some aspects, the nucleic acids have non-phosphorous backbone analogues such as sulfamate, 3′-thioformacetal, methylene (methylimino) (MMI), 3′-N-carbamate, or morpholino carbamate.

In some aspects, the anionic payload is a polynucleotide, also can be referred to as nucleotide or nucleic acid. The term “polynucleotide,” in its broadest sense, includes any compound and/or substance that comprise a polymer of nucleotides. Exemplary polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In some aspects, the synthetic polynucleotide is a synthetic messenger RNA (mRNA). As used herein, the term “messenger RNA” (mRNA) refers to any polynucleotide, which may be synthetic, which encodes a polypeptide, e.g., a G protein, and which is capable of being translated to produce the encoded polypeptide in vitro, in vivo, in situ or ex vivo.

The present disclosure expands the scope of functionality of traditional mRNA molecules by providing synthetic polynucleotides which comprise one or more structural and/or chemical modifications or alterations which impart useful properties to the polynucleotides including, in some aspects, the lack of a substantial induction of the innate immune response of a cell into which the polynucleotide is introduced. As used herein, a “structural” feature or modification is one in which two or more linked nucleotides are inserted, deleted, duplicated, inverted or randomized in a synthetic polynucleotide, primary construct or mmRNA without significant chemical modification to the nucleotides themselves. Because chemical bonds will necessarily be broken and reformed to effect a structural modification, structural modifications are of a chemical nature and hence are chemical modifications. However, structural modifications can result in a different sequence of nucleotides. For example, the polynucleotide “ATCG” may be chemically modified to “AT-5meC-G”. The same polynucleotide may be structurally modified from “ATCG” to “ATCCCG”.

In some aspects, the anionic payload can include untranslated regions. Untranslated regions (UTRs) of a gene are transcribed but not translated. The 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides, primary constructs and/or mmRNA of the present invention to enhance the stability of the molecule. The specific features can also be incorporated to ensure controlled down-regulation of the transcript in case they are misdirected to undesired organs sites.

It should be understood that those listed are examples and that any UTR from any gene may be incorporated into the respective first or second flanking region of the primary construct. Furthermore, multiple wild-type UTRs of any known gene can be utilized. It is also within the scope of the present invention to provide artificial UTRs which are not variants of wild type genes. These UTRs or portions thereof can be placed in the same orientation as in the transcript from which they were selected or may be altered in orientation or location. Hence a 5′ or 3′ UTR can be inverted, shortened, lengthened, made chimeric with one or more other 5′ UTRs or 3′ UTRs. As used herein, the term “altered” as it relates to a UTR sequence, means that the UTR has been changed in some way in relation to a reference sequence. For example, a 3′ or 5′ UTR can be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some aspects, the nucleotide also include a poly A tail, a miRNA binding site, an AU element, or any combination thereof.

In some aspects, an anionic payload having about 100 to about 1000 nucleotides in length can be any protein or fragments thereof having about 30 amino acids or less in length. For example, an anionic payload having about 100 to about 1000 nucleotides in length encodes PAPD5/7 (0.8 kb), or any fragments thereof.

In some aspects, an anionic payload having about 1000 to about 2000 nucleotides in length can be any protein or fragments thereof having about 660 amino acids or less in length. For example, an anionic payload having about 1000 to about 2000 nucleotides in length can encode G protein (1.5 kb).

In some aspects, an anionic payload having about 2000 to about 3000 nucleotides in length can be any protein or fragments thereof having about 1000 amino acids or less in length. For example, an anionic payload having about 2000 to about 3000 nucleotides in length can encode Gephyrin, protein anchor (2.2 kb).

In some aspects, an anionic payload having about 3000 to about 4000 nucleotides in length can be any protein or fragments thereof having about 1330 amino acids or less in length. For example, an anionic payload having about 3000 to about 4000 nucleotides in length can encode MDA5 (IFIH1) (3.0 kb).

i. Chemically Modified Polynucleotides

In some aspects, a polynucleotide of the present disclosure (e.g., an mRNA) comprises at least one chemically modified nucleoside and/or nucleotide. When the polynucleotides of the present disclosure are chemically modified, the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”).

A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target gene or protein, reduced non-specific binding to other gene or other molecules, as compared to an unmodified polynucleotide.

In some aspects, a polynucleotide of the present disclosure is chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., an mRNA) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In another aspect, the polynucleotide of the present disclosure (e.g., an mRNA) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine, or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”'s would be substituted for “U”s. For example, TD's of the present disclosure can be administered as RNAs, as DNAs, or as hybrid molecules comprising both RNA and DNA units.

In some aspects, the polynucleotide (e.g., an mRNA) includes a combination of at least two (e.g., 2, 3, 4, 5, 6, 7, 8, 8, 10, 11, 12, 13, 14, 15, 16, 17, 18, 18, 20 or more) modified nucleobases.

### 1. Base Modifications

In certain aspects, the chemical modification is at nucleobases in a polynucleotide of the present disclosure (e.g., an mRNA). In some aspects, the at least one chemically modified nucleoside is a modified uridine (e.g., pseudouridine (v), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., an mRNA) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

### 2. Backbone Modifications

In some aspects, the payload can comprise a “polynucleotide of the present disclosure” (for example comprising an mRNA), wherein the polynucleotide includes any useful modification to the linkages between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

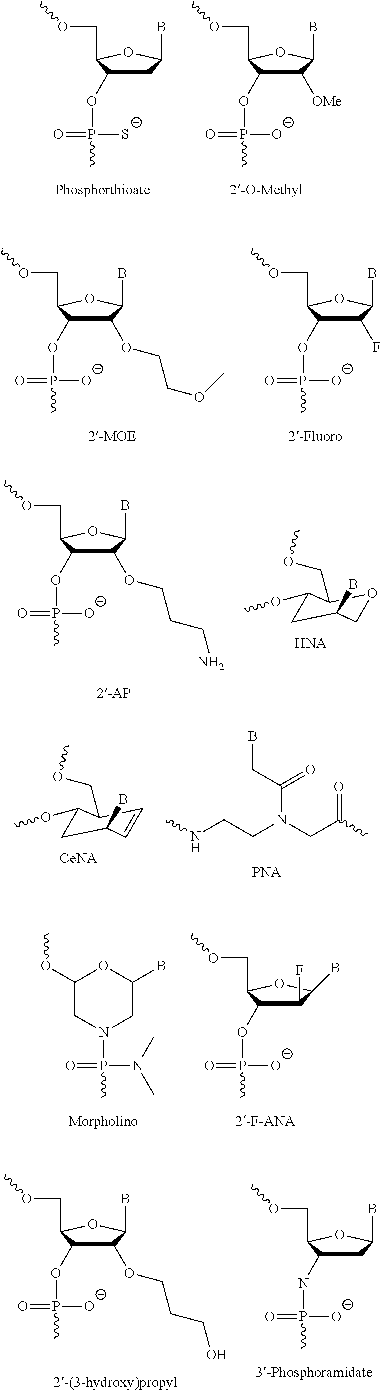

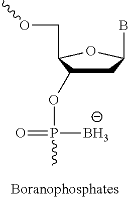

In some aspects, the presence of a backbone linkage disclosed above increase the stability (e.g., thermal stability) and/or resistance to degradation (e.g., enzyme degradation) of a polynucleotide of the present disclosure (e.g., an mRNA). In some aspects, the stability and/or resistance to degradation increases by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in the modified polynucleotide compared to a corresponding polynucleotide without the modification (reference or control polynucleotide)

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure ((e.g., an mRNA) are modified (e.g., all of them are phosphorothioate).

In some aspects, 1, 2, 3, 4, 5, 6, 7, 8, 9, 10, 11, 12, 13, 14, 15, 16, 17, 18, 19, 20, or 21 backbone linkages in a polynucleotide of the present disclosure (e.g., an mRNA) are modified (e.g., phosphorothioate).

In some aspects, the backbone comprises linkages selected from the group consisting of phosphodiester linkage, phosphotriesters linkage, methylphosphonate linkage, phosphoramidate linkage, phosphorothioate linkage, and combinations thereof.

### 3. Sugar Modifications

The modified nucleosides and nucleotides which can be incorporated into a polynucleotide of the present disclosure (e.g., an mRNA), can be modified on the sugar of the nucleic acid. Thus, in some aspects, the payload comprises a nucleic acid, wherein the nucleic comprises at least one nucleoside analog (e.g., a nucleoside with a sugar modification).

Incorporating affinity-enhancing nucleotide analogues in the polynucleotide, such as LNA or 2′-substituted sugars can allow the length of polynucleotide to be reduced, and also may reduce the upper limit of the size a polynucleotide before non-specific or aberrant binding takes place.

In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% of the nucleotides in a polynucleotide of the present disclosure (e.g., an mRNA) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a polynucleotide of the present disclosure (e.g., an mRNA) are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′->2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleoside analogues present in a polynucleotide of the present disclosure (e.g., an mRNA) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof.

In some aspects, nucleoside analogs present in a polynucleotide of the present disclosure comprise Locked Nucleic Acid (LNA); 2′-O-alkyl-RNA; 2′-amino-DNA; 2′-fluoro-DNA; arabino nucleic acid (ANA); 2′-fluoro-ANA, hexitol nucleic acid (HNA), intercalating nucleic acid (INA), constrained ethyl nucleoside (cEt), 2′-O-methyl nucleic acid (2′-OMe), 2′-0-methoxyethyl nucleic acid (2′-MOE), or any combination thereof.

### III. Methods of Making

EVs (e.g., exosomes) of the present disclosure can be produced by chemical synthesis, recombinant DNA technology, biochemical or enzymatic fragmentation of larger molecules, combinations of the foregoing or by any other method. In one aspect, the present disclosure provides a method of attaching a biologically active molecule to an EV (e.g., exosome) via an optimized link disclosed herein, e.g., via solid phase synthesis or conjugation.

In some aspects, EVs disclosed herein (e.g., exosomes) can be produced from a cell grown in vitro or a body fluid of a subject. When exosomes are produced from in vitro cell culture, various producer cells, e.g., HEK293 cells, CHO cells, and mesenchymal stem cells (MSCs), can be used. In some aspects, the producer cell is mesenchymal stem cells. In some aspects, a producer cell is not a dendritic cell, macrophage, B cell, mast cell, neutrophil, Kupffer-Browicz cell, cell derived from any of these cells, or any combination thereof.

### IV. Pharmaceutical Compositions

The present disclosure also provides pharmaceutical compositions comprising extracellular vesicles of the present disclosure that are suitable for administration to a subject. As discussed above, extracellular vesicles of the present disclosure can be homogeneous (i.e., all extracellular vesicles comprises the same type of biologically active molecules, with the same targeting moiety). However, in other aspects, the extracellular vesicles can comprise multiple targeting moieties, multiple payloads, etc.

The pharmaceutical compositions generally comprise an extracellular vesicle of the present disclosure and a pharmaceutically-acceptable excipient or carrier in a form suitable for administration to a subject. Pharmaceutically acceptable excipients or carriers are determined in part by the particular composition being administered, as well as by the particular method used to administer the composition.

There is a wide variety of suitable formulations of pharmaceutical compositions comprising extracellular vesicles of the present disclosure (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises one or more extracellular vesicles described herein.

In certain aspects, the extracellular vesicles described herein are co-administered with one or more additional therapeutic agents, in a pharmaceutically acceptable carrier. In some aspects, the pharmaceutical composition comprising the extracellular vesicles described herein is administered prior to administration of the additional therapeutic agent(s). In other aspects, the pharmaceutical composition comprising the extracellular vesicles described herein is administered after the administration of the additional therapeutic agent(s). In further aspects, the pharmaceutical composition comprising the extracellular vesicles described herein is administered concurrently with the additional therapeutic agent(s).

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles disclosed herein, use thereof in the compositions is contemplated.

Supplementary therapeutic agents can also be incorporated into the compositions of the present disclosure. Typically, a pharmaceutical composition is formulated to be compatible with its intended route of administration. The extracellular vesicles described herein can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition extracellular vesicles described herein is administered intravenously, e.g. by injection. The extracellular vesicles described herein can optionally be administered in combination with other therapeutic agents that are at least partly effective in treating the disease, disorder or condition for which the extracellular vesicles described herein are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water-soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Pharmaceutical compositions of the present disclosure can be sterilized by conventional, well known sterilization techniques. Aqueous solutions can be packaged for use or filtered under aseptic conditions and lyophilized, the lyophilized preparation being combined with a sterile aqueous solution prior to administration.

Sterile injectable solutions can be prepared by incorporating the extracellular vesicles described herein in an effective amount and in an appropriate solvent with one or a combination of ingredients enumerated herein, as desired. Generally, dispersions are prepared by incorporating the extracellular vesicles described herein into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The extracellular vesicles described herein can be administered in the form of a depot injection or implant preparation which can be formulated in such a manner to permit a sustained or pulsatile release of the extracellular vesicles described herein.

Systemic administration of compositions comprising extracellular vesicles described herein can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising extracellular vesicles described herein is administered intravenously into a subject that would benefit from the pharmaceutical composition. In certain aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the extracellular vesicles described herein and optionally a pharmaceutically active or therapeutic agent. The therapeutic agent can be a biological agent, a small molecule agent, or a nucleic acid agent.

Dosage forms are provided that comprise extracellular vesicles described herein. In some aspects, the dosage form is formulated as a liquid suspension for intravenous injection.

The extracellular vesicles disclosed herein or pharmaceutical composition comprising the extracellular vesicles may be used concurrently with other drugs. To be specific, the extracellular vesicles or pharmaceutical compositions of the present disclosure may be used together with medicaments such as hormonal therapeutic agents, chemotherapeutic agents, immunotherapeutic agents, medicaments inhibiting the action of cell growth factors or cell growth factor receptors and the like.

### V. Methods of Treatment and Use

The present disclosure also provides methods of treating a disease or condition in a subject in need thereof comprising administering an extracellular vesicle of the present disclosure or a combination thereof to the subject, e.g., a mammal, such as human subject. In some aspects, the present disclosure provides a method of treating a neurodegenerative disorder or cancer in a subject in need thereof, comprising administering to the subject a therapeutically effective amount of an extracellular vesicle of the present disclosure, or a pharmaceutical composition of the present disclosure.

In some aspects, the extracellular vesicles of the present disclosure can administered via intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In some aspects, the extracellular vesicles of the present disclosure can be used concurrently with other medicaments or treatment suitable for the treatment of the diseases and conditions disclosed herein.

The present disclosure also provides methods to delivering a payload to a target, comprising incorporating the payload and the targeting moiety into a EV of the present disclosure.

The present disclosure also provides methods to increase the resistance of a payload to degradation (e.g., nuclease-mediated degradation), comprising incorporating the payload, e.g., an anionic payload such as a nucleic acid (e.g., an antimir) into an extracellular vesicle of the present disclosure.

In some aspects, the present disclosure provides methods of crossing blood brain barrier (BBB) comprising administering the extracellular vesicles disclosed herein, e.g., extracellular vesicles comprising phenylalanine, tryptophan and/or tyrosine as a targeting moiety. An extracellular vesicle of the present disclosure loaded with anti-miRNA can be targeted to a BBB receptor, e.g., LAT1, as disclosed above. Once the extracellular vesicle is translocated across the BBB via receptor mediate transcytosis and undergoes cellular uptake by brain cells (e.g., neurons, astrocytes or microglia), the payload (e.g., an antimir) would be released and interact with an intracellular target (e.g., the antimir can bind to a target microRNA and trigger RNAse H mediated degradation).

In some aspects, encapsulation of the payload in an extracellular vesicle of the present disclosure can increase the resistance of the payload to degradation at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the free payload (i.e., not in an extracellular vesicle, e.g., free in solution).

In some aspects, encapsulation of the payload in an extracellular vesicle of the present disclosure can increase the resistance of the payload to degradation at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

The present disclosure also provides methods to increase the stability of a payload during administration (e.g., while in the subject's bloodstream) comprising incorporating the payload, e.g., an anionic payload such as a nucleic acid (e.g., an antimir) into an extracellular vesicle of the present disclosure.

In some aspects, encapsulation of the payload in an extracellular vesicle of the present disclosure can increase the stability (e.g., increase the resistance to nucleases) of the payload at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the free payload.

In some aspects, encapsulation of the payload in an extracellular vesicle of the present disclosure can increase the stability (e.g., increase the resistance to nucleases) of the payload at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

The present disclosure also provides methods to increase a payload's plasma half-life comprising incorporating the payload, e.g., an anionic payload such as a nucleic acid (e.g., an antimir) into an extracellular vesicle of the present disclosure.

In some aspects, encapsulation of the payload in an extracellular vesicle of the present disclosure can increase the plasma half-life of the payload at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, at least about 1900%, or at least about 2000%, compared to the free payload.

In some aspects, encapsulation of the payload in an extracellular vesicle of the present disclosure can increase the plasma half-life of the payload at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

In some aspects, the encapsulated payload is an antimir disclosed herein, e.g., an antisense oligonucleotide of SEQ ID NO: 25, or a variant or derivative thereof (e.g., an oligonucleotide having at least about 70% identity to the antisense oligonucleotide of SEQ ID NO: 25) wherein the encapsulation of the antimir in an extracellular vesicle of the present disclosure increases the plasma half-life of the antimir at least about 10-fold, at least about 12-fold, at least about 14-fold, at least about 16-fold, at least about 18-fold, or at least about 20-fold compared to the plasma half-life of the free antimir. In one particular aspect, the encapsulated payload is an antimir disclosed herein, e.g., an antisense oligonucleotide of SEQ ID NO: 25, or a variant or derivative thereof (e.g., an oligonucleotide having at least about 70% identity to the antisense oligonucleotide of SEQ ID NO: 25) wherein the encapsulation of the antimir in an extracellular vesicle of the present disclosure increases the plasma half-life of the antimir at least about 20-fold compared to the plasma half-life of the free antimir. In some aspects, the plasma half-life of the antimir encapsulated in an extracellular vesicle of the present disclosure is at least about 30 minutes, at least about 40 minutes, at least about 50 minutes, at least about 60 minutes, at least about 70 minutes, at least about 80 minutes, at least about 90 minutes, at least about 100 minutes, or at least about 120 minutes. In one particular aspects, the plasma half-life of the antimir (e.g., an antisense oligonucleotide of SEQ ID NO: 25) encapsulated in an extracellular vesicle of the present disclosure is at least about 90 minutes.

The present disclosure also provides methods to increase the permeation, delivery, transit, or transport of a payload through a physiological barrier, e.g., the BBB or the plasma membrane, comprising incorporating the payload, e.g., an anionic payload such as a nucleic acid (e.g., an antimir) into an extracellular vesicle of the present disclosure.

In some aspects, encapsulation of a payload in an extracellular vesicle of the present disclosure can increase the permeation, delivery, transit, or transport of the payload through a physiological barrier, e.g., the BBB or the plasma membrane, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% compared to the free payload.

In some aspects, encapsulation of a payload in an extracellular vesicle of the present disclosure can increase the permeation, delivery, transit, or transport of the payload through a physiological barrier, e.g., the BBB or the plasma membrane, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 21-fold, at least about 22-fold, at least about 23-fold, at least about 24-fold, at least about 25-fold, at least about 26-fold, at least about 27-fold, at least about 28-fold, at least about 29-fold, or at least about 30-fold compared to the free payload.

In some aspects, the extracellular vesicles of the present disclosure can be used to target stem cells, e.g., to deliver therapeutic molecules (e.g., therapeutic polynucleotides) or gene therapy components. In other aspects, the extracellular vesicles of the present disclosure can be used to treat cancer. For example, extracellular vesicles of the present disclosure can target a marker specific for a certain type of cancer, e.g., a glioma, breast cancer, pancreatic cancer, liver cancer, skin cancer, or cervical cancer, and carry as payload a therapeutic molecule (e.g., a therapeutic polynucleotide, a peptide, or a small molecule).

In specific aspects, the extracellular vesicles of the present disclosure can be used to treat pancreatic cancer.

In some aspects, the extracellular vesicles of the present disclosure can be used to treat or ameliorate the symptoms of a neurodegenerative disease, e.g., Alzheimer's disease. In some aspects, the extracellular vesicles of the present disclosure comprise a payload, e.g., an antimir, targeting a molecule overexpressed in Alzheimer's disease neuronal tissue, e.g., miRNA-485-3p. Accordingly, in some aspects, the administration of an extracellular vesicle of the present disclosure (e.g., an extracellular vesicle comprising a LAT1 targeting moiety to effectively transport the extracellular vesicle across the BBB and an antimir payload targeting miRNA-485-3p) to an Alzheimer's disease patient can prevent or ameliorate symptoms of Alzheimer's disease such as apoptosis, loss of mitochondrial function, or inflammation.

In some aspects, the present disclosure provides a method to reduce inflammation, e.g., neuroinflammation, in a subject suffering from a neurodegenerative disease (e.g., Alzheimer's disease) comprising administering to the subject a therapeutically effective amount of an extracellular vesicle of the present disclosure, wherein the extracellular vesicle comprises an therapeutic agent capable of effectively reducing inflammation, e.g., neuroinflammation, in the subject. In some aspects, the neuroinflammation is cortex inflammation. In some aspects, the neuroinflammation is hippocampus inflammation. In some aspects, the therapeutic agent is an antimir targeting miRNA-485-3p (e.g., an antimir of SEQ ID NO: 25 or fragment or variant thereof) wherein the antimir can reduce the levels of miRNA-485-3p in the subject.

In some aspects, the administration of an extracellular vesicle of the present disclosure to a subject suffering from a neurodegenerative disease (e.g., Alzheimer's disease) can decrease the level of neuroinflammation by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the neuroinflammation compared to the level of neuroinflammation observed in a subject or a population of subjects not treated with an extracellular vesicle of the present disclosure.

In some aspects, the present disclosure provides a method to reduce amyloid plaque burden in a subject suffering from Alzheimer's disease comprising administering to the subject a therapeutically effective amount of an extracellular vesicle of the present disclosure, wherein the extracellular vesicle comprises an therapeutic agent capable of effectively reducing amyloid plaque burden in the subject. In some aspects, the therapeutic agent is an antimir targeting miRNA-485-3p (e.g., an antimir of SEQ ID NO: 25 or fragment or variant thereof) wherein the antimir can reduce the levels of miRNA-485-3p in the subject.

In some aspects, the administration of an extracellular vesicle of the present disclosure to a subject suffering from a neurodegenerative disease (e.g., Alzheimer's disease) can decrease at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the amyloid plaque burden in the subject compared to the amyloid plaque burden observed in a subject or a population of subjects not treated with an extracellular vesicle of the present disclosure.

In some aspects, the present disclosure provides a method to recover and/or induce neurogenesis in a subject suffering from a neurodegenerative disease (e.g., Alzheimer's disease) comprising administering to the subject a therapeutically effective amount of an extracellular vesicle of the present disclosure, wherein the extracellular vesicle comprises a therapeutic agent capable of effectively recovering and/or inducing neurogenesis in the subject. In some aspects, the therapeutic agent is an antimir targeting miRNA-485-3p (e.g., an antimir of SEQ ID NO: 25 or fragment or variant thereof) wherein the antimir can reduce the levels of miRNA-485-3p in the subject.

In some aspects, the administration of an extracellular vesicle of the present disclosure to a subject suffering from a neurodegenerative disease (e.g., Alzheimer's disease) can recover and/or induce neurogenesis in the subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the level of neurogenesis observed in a subject or a population of subjects not treated with an extracellular vesicle of the present disclosure.

In some aspects, the present disclosure provides a method to improve cognitive function in a subject suffering from a neurodegenerative disease (e.g., Alzheimer's disease) comprising administering to the subject a therapeutically effective amount of an extracellular vesicle of the present disclosure, wherein the extracellular vesicle comprises an therapeutic agent capable of effectively improving cognitive function in the subject. In some aspects, the therapeutic agent is an antimir targeting miRNA-485-3p (e.g., an antimir of SEQ ID NO: 25 or fragment or variant thereof) wherein the antimir can reduce the levels of miRNA-485-3p in the subject.

In some aspects, the administration of an extracellular vesicle of the present disclosure to a subject suffering from a neurodegenerative disease (e.g., Alzheimer's disease) can increase the cognitive function of the subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the cognitive function observed in a subject or a population of subjects not treated with an extracellular vesicle of the present disclosure.

### VI. Kits

The present disclosure also provides kits, or products of manufacture, comprising an extracellular vesicle or a pharmaceutical composition of the present disclosure and optionally instructions for use. In some aspects, the kit or product of manufacture comprises an extracellular vesicle or a pharmaceutical composition of the present disclosure in one or more containers. In some aspects, the kit or product of manufacture comprises an extracellular vesicle or a pharmaceutical composition of the present disclosure and a brochure. In some aspects, the kit or product of manufacture comprises an extracellular vesicle or a pharmaceutical composition of the present disclosure and instructions for use. One skilled in the art will readily recognize that an extracellular vesicle or a pharmaceutical composition of the present disclosure, or combinations thereof, can be readily incorporated into one of the established kit formats which are well known in the art.

In some aspects, the kit or product of manufacture comprises an extracellular vesicle the present disclosure in dry form in a container (e.g., a glass vial), and optionally a vial with a solvent suitable to hydrate the dry the cationic carrier unit, and optionally instructions for the hydration of the extracellular vesicle and the formation of extracellular vesicles. In some aspects, the kit or product of manufacture further comprises at least one additional container (e.g., a glass vial) with the extracellular vesicle's anionic payload (e.g., an antisense oligonucleotide). In some aspects, the kit or product of manufacture comprises an extracellular vesicle of the present disclosure in a dry form and the extracellular vesicle's anionic payload also in dry form in the same container, or in different containers. In some aspects, the kit or product of manufacture comprises an extracellular vesicle of the present disclosure in solution and the extracellular vesicle's anionic payload also in solution in the same container, or in different containers. In some aspects, the kit or product of manufacture comprises an extracellular vesicle of the present disclosure in solution, and instructions for use. In some aspects, the kit or product of manufacture comprises an extracellular vesicle of the present disclosure in dry form, and instructions for use (e.g., instructions for reconstitution and administration).

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986)); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All publications, patent applications, patents, and other references mentioned herein are incorporated by reference in their entirety. Database entries and electronic publications disclosed in the present disclosure are incorporated by reference in their entireties. The version of the database entry or electronic publication incorporated by reference in the present application is the most recent version of the database entry or electronic publication that was publicly available at the time the present application was filed. The database entries corresponding to gene or protein identifiers (e.g., genes or proteins identified by an accession number or database identifier of a public database such as Genbank, Refseq, or Uniprot) disclosed in the present application are incorporated by reference in their entireties. The gene or protein-related incorporated information is not limited to the sequence data contained in the database entry. The information incorporated by reference includes the entire contents of the database entry in the most recent version of the database that was publicly available at the time the present application was filed. In case of conflict, the present specification, including definitions, will control. In addition, the materials, methods, and examples are illustrative only and not intended to be limiting.

### Examples

The following examples are provided for illustrative purposes only, and are not to be construed as limiting the scope or content of the invention in any way. The practice of the current invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods in Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

1. An extracellular vesicle (EV) comprising a targeting moiety via an optional linker, wherein the targeting moiety is capable of being transported by large neutral amino acid transporter 1 (LAT1), wherein the extracellular vesicle optionally further comprises a biologically active molecule that is linked to the exterior surface of the EV or to the interior surface of the EV or is inside of the EV.

2-5. (canceled)

6. The extracellular vesicle of claim 1, wherein the biologically active molecule comprises a polypeptide, a peptide, a polynucleotide (DNA and/or RNA), a chemical compound, a nucleic acid, an antibody or an antigen binding fragment thereof, a fusion protein, or any combination thereof.

7-9. (canceled)

10. The extracellular vesicle of claim 6, wherein the nucleic acid comprises a mRNA, a miRNA, a miRNA sponge, a tough decoy miRNA (TD), an antimir (antagomir), a small RNA, a rRNA, a siRNA, a shRNA, a gDNA, a cDNA, a pDNA, a PNA, a BNA, an antisense oligonucleotide (ASO), an aptamer, a cyclic dinucleotide, a phosphorodiamidate morpholino oligonucleotide (PMO), or any combination thereof.

11-13. (canceled)

14. The extracellular vesicle of claim 10, wherein the miRNA is miR-485-3p, miR-204-5p, or miR-132-3p.

15. (canceled)

16. The extracellular vesicle of claim 10, wherein the nucleic acid comprises: 5′-UGUAUGA-3′, 5′-GUGUAUGA-3′, 5′-CGUGUAUGA-3′, 5′-CCGUGUAUGA-3′ (SEQ ID NO: 2), 5′-GCCGUGUAUGA-3′ (SEQ ID NO: 3), 5′-AGCCGUGUAUGA-3′ (SEQ ID NO: 4), 5′-GAGCCGUGUAUGA-3′ (SEQ ID NO: 5), 5′-AGAGCCGUGUAUGA-3′ (SEQ ID NO: 6), 5′-GAGAGCCGUGUAUGA-3′ (SEQ ID NO: 7), 5′-GGAGAGCCGUGUAUGA-3′ (SEQ ID NO: 8), 5′-AGGAGAGCCGUGUAUGA-3′ (SEQ ID NO: 9), 5′-GAGGAGAGCCGUGUAUGA-3′ ID (SEQ ID NO: 10), 5′-AGAGGAGAGCCGUGUAUGA-3′ (SEQ ID NO: 11), 5′-GAGAGGAGAGCCGUGUAUGA-3′ (SEQ ID NO: 12); 5′-UGUAUGAC-3′, 5′-GUGUAUGAC-3′, 5′-CGUGUAUGAC-3′ (SEQ ID NO: 13), 5′-CCGUGUAUGAC-3′ (SEQ ID NO: 14), 5′-GCCGUGUAUGAC-3′ (SEQ ID NO: 15), 5′-AGCCGUGUAUGAC-3′ (SEQ ID NO: 16), 5′-GAGCCGUGUAUGAC-3′ (SEQ ID NO: 17), 5′-AGAGCCGUGUAUGAC-3′ (SEQ ID NO: 18), 5′-GAGAGCCGUGUAUGAC-3′ (SEQ ID NO: 19), 5′-GGAGAGCCGUGUAUGAC-3′ (SEQ ID NO: 20), 5′-AGGAGAGCCGUGUAUGAC-3′ (SEQ ID NO: 21), 5′-GAGGAGAGCCGUGUAUGAC-3′ (SEQ ID NO: 22), 5′-AGAGGAGAGCCGUGUAUGAC-3′ (SEQ ID NO: 23), 5′-GAGAGGAGAGCCGUGUAUGAC-3′ (SEQ ID NO: 24), 5′-AGAGAGGAGAGCCGUGUAUGAC-3′ (SEQ ID NO: 25), 5′-TGTATGA-3′, 5′-GTGTATGA-3′, 5′-CGTGTATGA-3′, 5′-CCGTGTATGA-3′ (SEQ ID NO: 26), 5′-GCCGTGTATGA-3′ (SEQ ID NO: 27), 5′-AGCCGTGTATGA-3′ (SEQ ID NO: 28), 5′-GAGCCGTGTATGA-3′ (SEQ ID NO: 29), 5′-AGAGCCGTGTATGA-3′ (SEQ ID NO: 30), 5′-GAGAGCCGTGTATGA-3′ (SEQ ID NO: 31), 5′-GGAGAGCCGTGTATGA-3′ (SEQ ID NO: 32), 5′-AGGAGAGCCGTGTATGA-3′ (SEQ ID NO: 33), 5′-GAGGAGAGCCGTGTATGA-3′ (SEQ ID NO: 34), 5′-AGAGGAGAGCCGTGTATGA-3′ (SEQ ID NO: 35), 5′-GAGAGGAGAGCCGTGTATGA-3′ (SEQ ID NO: 36); 5′-TGTATGAC-3′, 5′-GTGTATGAC-3′, 5′-CGTGTATGAC-3′ (SEQ ID NO: 37), 5′-CCGTGTATGAC-3′ (SEQ ID NO: 38), 5′-GCCGTGTATGAC-3′ (SEQ ID NO: 39), 5′-AGCCGTGTATGAC-3′ (SEQ ID NO: 40), 5′-GAGCCGTGTATGAC-3′ (SEQ ID NO: 41), 5′-AGAGCCGTGTATGAC-3′ (SEQ ID NO: 42), 5′-GAGAGCCGTGTATGAC-3′ (SEQ ID NO: 43), 5′-GGAGAGCCGTGTATGAC-3′ (SEQ ID NO: 44), 5′-AGGAGAGCCGTGTATGAC-3′ (SEQ ID NO: 45), 5′-GAGGAGAGCCGTGTATGAC-3′ (SEQ ID NO: 46), 5′-AGAGGAGAGCCGTGTATGAC-3′ (SEQ ID NO: 47), 5′-GAGAGGAGAGCCGTGTATGAC-3′ (SEQ ID NO: 48), or 5′-AGAGAGGAGAGCCGTGTATGAC-3′ (SEQ ID NO: 49).

17-22. (canceled)

23. The extracellular vesicle of claim 10, wherein the nucleic acid comprises at least one modified nucleotide, which comprises a locked nucleic acid (LNA), an unlocked nucleic acid (UNA), an arabino nucleic acid (ABA), a bridged nucleic acid (BNA), and/or a peptide nucleic acid (PNA).

24. (canceled)

25. The extracellular vesicle of claim 10, wherein the nucleic acid comprises a backbone modification, which comprises a phosphorodiamidate morpholino oligomer (PMO) and/or phosphorothioate (PS) modification.

26. (canceled)

27. The extracellular vesicle of claim 6, wherein the nucleic acid targets a transcript.

28. The extracellular vesicle of claim 6, wherein the nucleic acid is linked to the EV via a linker.

29. (canceled)

30. The extracellular vesicle of claim 6, wherein the antigen binding fragment thereof comprises scFv, (scFv)2, Fab, Fab′, F(ab′)2, F(ab1)2, Fv, dAb, and Fd fragment, diabodys, antibody-related polypeptide, or any fragment thereof.

31. The extracellular vesicle of claim 1, wherein the targeting moiety is an amino acid.

32. (canceled)

33. The extracellular vesicle of claim 1, wherein the targeting moiety is an amino acid comprising phenylalanine, tryptophan, tyrosine, valine, leucine, and/or isoleucine.

34-36. (canceled)

37. The extracellular vesicle of claim 1, wherein the EV further comprises an exosomal protein, wherein the targeting moiety is optionally linked to the exosomal protein.

38. (canceled)

39. The extracellular vesicle of claim 1, wherein the EV is an exosome.

40. (canceled)

41. A pharmaceutical composition comprising the extracellular vesicle of claim 1 and a pharmaceutically acceptable carrier.

42. A kit comprising the EV of claim 1 and instructions for use.

43. A cell that produces the extracellular vesicle of claim 1.

44. A method of producing the extracellular vesicle comprising culturing the cell of claim 43 in suitable condition.

45. A method of treating or preventing a disease or disorder in a subject in need thereof comprising administering the EV of claim 1 to the subject.

46. The method of claim 45, wherein the disease or disorder is a cancer, an inflammatory disorder, a neurodegenerative disorder, a central nervous disease, or a metabolic disease.

47. (canceled)
